SEQUENCE LISTING (6 sequences)

SEQ ID NO: 1 - protein, 107 aa, H. sapiens
YAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIH

SEQ ID NO: 2 - protein, 91 aa, H. sapiens
MHPEPAPPPSRSSPELPPSGGSTTSGSRRSRRRSGDGEPPGAPPPPPSAVTYPDWIGQSYSEVMSLNEHSMQALSWRKLYLSRAKLKASSR

SEQ ID NO: 3 - protein, 44 aa, H. sapiens
SAVTYPDWIGQSYSEVMSLNEHSMQALSWRKLYLSRAKLKASSR

SEQ ID NO: 4 - protein, 28 aa, H. sapiens
MSLNEHSMQALSWRKLYLSRAKLKASSR

SEQ ID NO: 5 - protein, 36 aa, H. sapiens
TCILPNIEAVSNVHNLNSVKESPHERMHRHIELAWA

SEQ ID NO: 6 - protein, 46 aa, H. sapiens
HFYRSLVSHKTDRQFQELNELAEFARLQDQLDHRGDHPLTPGSHYA

(19) United States
(12) Patent Application Publication
(10) Pub. No.: US 2012/0165265 A1
(43) Pub. Date: Jun. 28, 2012

(54) Calcium Signaling Modulators Involving STIM and ORAI Proteins

(76) Inventors: Ricardo E. Dolmetsch, Stanford, CA (US); Richard Sheridan Lewis, Stanford, CA (US)

(21) Appl. No.: 13/203,700

(22) PCT Filed: Feb. 26, 2010
(86) PCT No.: PCT/US10/25529

§ 371 Date: Mar. 19, 2012

Related U.S. Application Data
(60) Provisional application No. 61/155,761, filed on Feb. 26, 2009.

(51) Int. Cl. A61K 38/17; C12N 5/071; C12N 5/0783; G01N 21/64; C40B 30/06; C12Q 1/66; G01N 33/566; A61P 43/00; C12Q 1/02

(52) U.S. Cl. 514/17.4; 435/375; 435/366; 435/29; 506/10; 435/8; 435/7.21

(57) ABSTRACT

The present invention provides compositions and methods for investigating the structural basis of the activation process of CRAC channels, which are essential for T-lymphocyte activation and adaptive immunity. The invention also provides compositions and methods to design, identify and evaluate agents that modulate calcium signaling by regulating the interaction between STIM and Orai proteins. The invention also provides therapeutic agents for cases of immunological disorders, compromised immune function, organ transplantation, or thrombosis.

### STATEMENT OF GOVERNMENTAL SUPPORT

This invention was made with U.S. Government support under National Institutes of Health Grants R01 NS048564 and R01 GM45374. The Government has certain rights in this invention.

### BACKGROUND OF THE INVENTION

Store-operated calcium channels (SOCs) comprise the major receptor-activated calcium entry pathway in non-excitable mammalian cells and play important roles in the control of gene expression, cell differentiation, secretion and calcium homeostasis. In their native environment, SOCs are activated by the stimulation of phospholipase C (PLC)-coupled receptors that generate inositol 1,4,5-trisphosphate (IP₃) and release Ca²⁺ from the endoplasmic reticulum (ER). The defining feature of SOCs is that they are activated by the reduction of calcium in the endoplasmic reticulum (ER) rather than by receptor-associated signaling molecules, such as G proteins, phospholipase C (PLC) or inositol 1,4,5-trisphosphate (IP₃).

The most extensively characterized store-operated channel is the calcium release-activated calcium (CRAC) channel, whose activation is a steep function of Ca²⁺ in the ER. CRAC channels play an essential role in the initiation of the adaptive immune response in T-lymphocytes as well as mast cells and are central to immunity in humans, as a single loss-of-function mutation in the CRAC channel leads to severe combined immunodeficiency. CRAC channels provide the pathway for Ca²⁺ entry triggered by antigen recognition or allergens, and are required for T-cell activation and mast cell degranulation.

The molecular mechanism by which ER Ca²⁺ depletion activates the CRAC channel has been a mystery since the original proposal of the store-operated calcium entry (SOCE) hypothesis. However, remarkable progress has been made in the past several years following the identification of STIM1 as the ER Ca²⁺ sensor and Orai1 as the pore-forming subunit of the CRAC channel. Recent studies show that the loss of ER Ca²⁺ triggers the oligomerization of STIM1 and its accumulation in regions of the ER located within 10-25 nm of the plasma membrane, commonly referred to as “puncta”.

Orai1 accumulates in overlying regions of the plasma membrane (PM) in register with STIM1, culminating in the local entry of Ca²⁺ through CRAC channels. A recent study shows that STIM1 oligomerization is the key event that triggers the redistribution of STIM1 and Orai1, translating changes in the Ca²⁺ concentration of the ER into graded activation of the CRAC channel.

STIM1 forms puncta in response to store depletion even when expressed in the nominal absence of Orai1, suggesting that its initial target may be independent of Orai1. In contrast, Orai1 only forms puncta in store-depleted cells when co-expressed with STIM1, suggesting that it becomes trapped at ER-PM junctions by binding to STIM1 or an associated protein. Several parts of the cytosolic domain of STIM1, including the C-terminal polybasic domain, an ERM-like domain, and a serine-proline-rich domain, have been implicated in the activation of Orai1, but their specific roles and interactions in these localization events are not understood.

The molecular mechanism by which STIM1 activates the CRAC channel has also been controversial. A widely considered ‘diffusible messenger’ model posits that STIM1 oligomerization promotes the synthesis of a ‘Ca²⁺ influx factor’ (CIF), which is delivered locally at ER-PM junctions to stimulate iPLA₂β to produce lysolipids that activate I_{CRAC}. An alternative ‘conformational coupling’ hypothesis proposes that STIM1 binds physically to the CRAC channel or to an associated protein to activate Ca²⁺ entry. Precisely how this binding event might activate the channel is unclear; one recent study has proposed that STIM1 links Orai1 dimers to form active tetrameric channels, while another study concludes that Orai1 is a tetramer at rest, suggesting instead that STIM1 activates Orai1 by an allosteric mechanism.

There is currently no definitive evidence demonstrating a direct interaction between STIM1 and Orai1. However, this information would be invaluable for designing, screening for and evaluating modulators of the interaction between STIM1 and Orai1 as agents to modulate calcium signaling through store-operated channels in a variety of systems including the immune system.

In addition, the adaptive immune response can be activated or inhibited by altering calcium influx in T-lymphocytes. An inhibition of the immune response could clinically be used to suppress organ rejection during transplantation and to normalize immune response in cases of immunological disorders such as autoimmune diseases, inflammation or hypersensitivity. An activation of the immune response would be desirable in cases of immunodeficiencies, where immune function is compromised. It would be beneficial for either case to develop agents that modulate calcium influx in T-lymphocytes.

Related publications include Li et al. (2007). J Biol Chem 282, 29448-29456; Liou, et al (2007) Proc Natl Acad Sci USA 104, 9301-9306; Liou et al (2005) Curr Biol 15, 1235-1241; Huang et al (2006). Nat Cell Biol 8, 1003-1010; Zhang et al. (2005) Nature 437, 902-905.

### SUMMARY OF THE INVENTION

Methods and compositions are provided for modulating the calcium influx mediated by CRAC channels, and in particular modulating calcium influx in T lymphocytes, thereby altering immune responses mediated by such T lymphocytes. In some embodiments of the invention, a CRAC channel is contacted with a polypeptide that inhibits opening of the CRAC channel. In other embodiments of the invention, a CRAC channel is contacted with a polypeptide that enhances opening of the CRAC channel. These inhibiting or enhancing polypeptides may be referred to as modulating polypeptides.

In some embodiments of the invention, an enhancing polypeptide consists or consists essentially of a minimal, highly conserved 107-amino acid residue STIM1 domain, referred to herein as CAD, that binds to Orai1 and opens the CRAC channel, and polypeptides that comprise, consist or consist essentially of fragments and derivatives thereof. Derivatives include polypeptides fused to a permeant domain, e.g. nona-arginine, tat, etc. as known in the art.

In some embodiments of the invention, an inhibiting polypeptide consists or consists essentially of an Orai domain that inhibits opening of the CRAC channel, and polypeptides that comprise, consist or consist essentially of fragments and derivatives thereof. Derivatives include polypeptides fused to a permeant domain, e.g. nona-arginine, tat, etc. as known in the art. Inhibiting polypeptides include, without limitation, Orai NT amino acids 1-91; Orai NT amino acids 48-91; Orai NT amino acids 64-91; Orai II-III amino acids 142-177; and Orai CT amino acids 256-301.

The modulating polypeptide and fragments and derivatives thereof are used in methods of opening CRAC channels, the methods comprising contacting a CRAC channel with a CAD polypeptide in an amount effective to open the channel. The channel may be present as an isolated polypeptide or on a cell, where the cell may be present in culture as an isolated cell or tissue, or in an in vivo environment. Polypeptides may be introduced into the cell by fusing the polypeptide to a permeant domain, and contacting the channel or cell comprising a channel with the fusion polypeptide. Alternatively an expression construct is introduced to a cell, where the modulating polypeptide is then expressed. Such methods provide for modulation of a variety of cellular processes, including T-lymphocyte activation and adaptive immunity, mast cell degranulation, platelet activation, and muscle development, and for screening methods that relate to such processes.

In some embodiments methods are provided for screening a candidate agent (e.g. a small molecule compound) for modulation of calcium signaling through store-operated channels by decreasing or enhancing the interaction between STIM and Orai proteins, where a candidate agent is brought into contact with a CRAC channel, and the effect on calcium signaling determined. A modulating polypeptide of the invention may be utilized as a control, or in competition assays. Also provided are compositions and methods related to the identification of a 107-residue CRAC activation domain (CAD), which facilitates direct binding of STIM1 to Orai1 and provides a molecular tool to further investigate the structural basis of the activation process of CRAC channels.

In one embodiment methods are provided for screening candidate agents that modulate calcium signaling through store-operated channels by modulating the interaction between STIM1 and Orai1 proteins, as characterized by enhanced or decreased CAD-Orai binding. For example, a candidate agent may be screened for the ability to interfere between the binding of CAD to Orai1.

In other embodiments methods are provided for screening candidate agents that modulate calcium signaling through store-operated channels by modulating the interaction between STIM and Orai proteins, as characterized by enhanced or decreased CAD-Orai binding.

The above summary is not intended to include all features and aspects of the present invention nor does it imply that the invention must include all features and aspects discussed in this summary.

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings are not to-scale.

FIG. 1. Deletion of the STIM1 Polybasic Domain Distinguishes Orai1-dependent and -independent Mechanisms of STIM1 Accumulation at ER-PM Junctions. (A) Wild-type mCh-STIM1 expressed alone in HEK 293 cells redistributes from a diffuse ER distribution (Rest) to the cell periphery after store depletion with TG (0 Ca+TG). After depletion, puncta are visible at the cell footprint. (B) eGFP-myc-Orai1 when expressed alone does not redistribute into puncta after depletion. (C) When expressed together, STIM1 and Orai1 form colocalized puncta after store depletion. (D) Unlike wild-type STIM1, STIM1-DK expressed alone fails to form puncta; however, when coexpressed with Orai1 both proteins form colocalized puncta after store depletion (E). (F) Store depletion activates I_{CRAC} in HEK 293 cells expressing STIM1-DK and Orai1 demonstrating that the polybasic region is not essential for I_{CRAC} activation. All images were taken of cell footprints using confocal microscopy. Scale bars, 10 μm.

FIG. 2. Identification of CAD as a Potent CRAC Channel Activator. (A) Full-length STIM1 with its putative functional domains (top) and truncated versions of STIM1 (D1-D9; bottom) with the CAD shown in gray. (B) NFAT-dependent luciferase activity in HEK 293T (NFAT-Luc) cells transfected with wild-type (WT) or truncated (D1-D9) STIM1 constructs. Cells were treated with 1 μM PMA or 1 μM TG+PMA as shown. D5 (CAD) is the minimal region that is necessary and sufficient to activate NFAT. Data are shown as mean±sem (n=4). (C) CAD activates SOCE without depleting intracellular Ca²⁺ stores. Mean [Ca²⁺]ᵢ (±sem) in untransfected HEK 293 cells (black; n=28) and cells expressing CAD+Orai1 (n=15), CAD+Orai1_{E106A} (n=17), or CAD (342-440)+Orai1 (n=12). (D) (left) I_{CRAC} develops slowly in a representative cell cotransfected with WT GFP-STIM1+myc-Orai1. Current recorded during brief pulses to −100 mV in 2 mM Ca²⁺ₒ is plotted against time after break-in. (right) Characteristic I-V relationship for I_{CRAC} recorded in 20 mM Ca²⁺ₒ from the same cell. (E) (left) I_{CRAC} is constitutively active in a representative cell cotransfected with YFP-CAD+myc-Orai1. Current is plotted as in D. (right) I-V relationship for the CAD-induced current recorded in 20 mM Ca²⁺ₒ from the same cell. (F) Current densities in cells transfected with CAD or Orai1 alone, or cotransfected with myc-Orai1 and GFP-STIM1, YFP-CAD, or YFP-CT-STIM1. Current measured at −100 mV during voltage ramps in 20 mM Ca²⁺ₒ was normalized to the cell capacitance. Mean values±sem from 4-5 cells are shown.

FIG. 3. CAD Associates with Orai1. (A) YFP-CAD is cytosolic when expressed by itself in a HEK 293 cell (top), but accumulates at the cell perimeter when coexpressed with Orai1 (bottom). Scale bar, 10 μm. (B, C) Western blots of cell lysates (left) or immunoprecipitated material (right) from cells expressing CAD, Orai1, or CAD+Orai1. Anti-Flag antibodies co-immunoprecipitate GFP-myc-Orai1 with Flag-myc-CAD (B), and co-immunoprecipitate YFP-CAD with Flag-myc-Orai1 (C). (D) CT-STIM1 does not co-immunoprecipitate with myc-Orai1 (representative of 4 experiments). (E) Schematic depiction of the yeast split ubiquitin assay. (F) CAD associates with Orai1 in the split ubiquitin assay. Yeast containing NubG-Orai1 and either Cub-LV alone or CAD-Cub-LV, grow well on plates lacking tryptophan and leucine but containing histidine (SD-TL). Only yeast expressing NubG-Orai1 and CAD-Cub-LV grow on plates lacking all three amino acids (SD-TLH) in the absence or presence of 5 mM 3-aminotriazole (3AT), a competitive HIS3 inhibitor that increases the stringency of selection. Yeast containing CAD-Cub-LV and NubG-Orai1 also activate LacZ whereas cells expressing NubG-Orai1 and Cub do not. The homomeric interaction of Alg5 is shown as a positive control.

FIG. 4. CAD Binds Directly to Orai1. (A) Glutathione beads precipitate EE-Orai1-His₈ with GST-CAD but not GST alone. (B) (Left) Size-exclusion profile of purified EE-Orai1-His₈. On SDS-PAGE (right), peak fractions contain EE-Orai1-His₈ as a pair of glycosylated and unglycosylated bands at ˜37 kDa. (C) (Left) Size-exclusion profile of EE-Orai1-His₈ and CAD-His₆ coexpressed in Hi5 cells and affinity purified in 0.5 M NaCl. Much of the CAD and Orai1 co-elute in the void volume (MW>10 MDa) of the Superose 6 gel filtration column (fraction 4). The band corresponding to the dimer of EE-Orai1-His₈ (arrow) was identified by Western blotting. (D) Multi-angle light scattering (MALS) indicates a molecular weight of ˜58 kDa for purified CAD-His₆, approximately four times the predicted mass of the monomer.

FIG. 5. CAD Binds to Both the N- and C-termini of Orai1. (A) In this split ubiquitin assay, b-gal production and growth of transformants on plates lacking histidine indicate a strong interaction between CAD and the C-terminus of Orai1, a weaker interaction with the N-terminus, and lack of interaction with the II-III loop of Orai1. (B) CAD in HEK 293T cells co-immunoprecipitates with YFP-tagged N-terminal and C-terminal fragments of Orai1 but not with the II-III loop. (C) Split ubiquitin assays showing that CAD interacts with aa 48-91 of the Orai1 N-terminus; this appears to be due to binding to aa 68-91 rather than aa 48-70. (D) CAD co-immunoprecipitates with Orai1-NT(48-91) but only very weakly with the entire N-terminus of Orai1 (aa 1-91). (E) Whole-cell recordings in 20 mM Ca²⁺ₒ from HEK 293 cells co-expressing truncated Orai1-GFP proteins and YFP-CAD. Deletion of the N- or C-terminus of Orai1 abrogates function, but a substantial level of I_{CRAC} is generated by Orai1-DN73. (F) Summary of I_{CRAC} measurements (current at −100 mV, 20 mM Ca²⁺ₒ, normalized to cell capacitance) with the indicated constructs (4 cells each, mean±sem).

FIG. 6. CAD Links Multiple CRAC Channels to Form Clusters. (A) Negative stain electron microscopy of purified EE-Orai1-His₈ (left; fraction 8 from FIG. 4B), or complexes of EE-Orai1-His₈ and CAD-His₆ (right panels; fractions 4 and 8 from FIG. 4C). Scale bars, 100 nm (top), 20 nm (bottom, enlargements from dashed boxes). (B) Quantitation of cluster sizes for each condition shown in A; Orai1 (n=174), Orai1+CAD (fr 8, n=134; fr 4, n=90). Each histogram is normalized to its maximum bin value. (C) FRAP of HEK 293 cells expressing eGFP-Orai1 (top row) or eGFP-Orai1+CAD (bottom row). Images are shown at the indicated times after bleaching a bar across the cell footprint (left). Scale bars, 5 μm. (D) Time course of FRAP from single cells expressing eGFP-Orai1 alone or with CAD. The superimposed fits indicate diffusion coefficients of 0.12 μm²/s (Orai1) and 0.026 μm²/s (Orai1+CAD). (E) Mean diffusion coefficients and mobile fractions of GFP-Orai1 expressed alone (n=9) or with CAD (n=6; means±sem).

FIG. 7. CRAC Channel Clustering and Activation by STIM1 are Separable and Require the CAD Region. (A) When coexpressed with Orai1, STIM1-DCAD fails to form puncta and cluster Orai1 after store depletion. In contrast, STIM1₁₋₄₄₈ (B), STIM1₁₋₄₄₀ (C), and STIM1 C437G (D) co-accumulate with Orai1 in puncta after store depletion. In A-D, images are confocal micrographs of the HEK 293 cell footprint. Scale bars, 10 μm. (E) Ca²⁺ measurements (mean±sem) in HEK 293 cells expressing the indicated constructs. In all cases, Orai1 was tagged with eGFP and the STIM1 or its variant was tagged with mCherry. (F) Puncta formation and Ca²⁺ influx in cells expressing Orai1 and STIM1 variants. Each set of bars shows the fraction of the cell's STIM1 or Orai1 fluorescence that is colocalized to puncta before and after store depletion (mean±sem, n=4 cells for each) and the initial rate of Ca²⁺ entry (dR/dt, where R is the fura-2 350/380 fluorescence ratio) measured 10-20 s after readdition of Ca²⁺ in E.

FIG. 8. Expression of truncated STIM1 constructs. (A) HEK 293T cells were transfected with plasmids encoding YFP-tagged WT or truncated STIM1 constructs (D1-9 correspond to the diagrams shown in FIG. 2) and analyzed by Western blotting with anti-GFP antibodies. The ability of each STIM1 construct to activate an NFAT-luciferase reporter gene in the experiments of FIG. 2 is shown below the blot. Representative of 3 experiments. (B) Western blot of HEK 293 cells expressing myc-Orai1 together with GFP-tagged WT-STIM1, YFP-tagged CT-STIM1 or YFP-CAD. The upper panel shows expression of STIM1 proteins while the lower panel shows the expression of the Orai1 proteins (representative of 3 experiments).

FIG. 9. Sequence alignment of the CRAC activation domain (CAD) of STIM homologs. STIM1 and STIM2 sequences from vertebrate and invertebrate species were aligned with CLC Sequence Viewer v5.0 using default settings for gap penalties. The degree of conservation for each position is shown in the bar graph at bottom. Colors are standard Rasmol colors. Two gaps have been introduced to accommodate the C. elegans sequence. Accession numbers: Pan troglodytes STIM1 (XP_{—}001160553.1), Homo sapiens STIM1 (NP_{—}003147.2), Macaca mulatta STIM1 (XP_{—}001112949.1), Bos taurus STIM1 (NP_{—}001030486.1), Equus caballus STIM1 (XP_{—}001499902.2), Canis familiaris STIM1 (XP_{—}850663.1), Rattus norvegicus STIM1 (NP_{—}001101966.2), Mus musculus STIM1 (NP_{—}033313.2), Sus scrofa STIM1 (NP_{—}001124446.1), Monodelphis domestica STIM1 (XP_{—}001370519.1), Gallus gallus STIM1 (NP_{—}001026009.1), Xenopus laevis STIM1 (NP_{—}001090506.1), Danio rerio (NP_{—}001038264.1), Tetraodon nigroviridis (CAF95381.1), Rattus norvegicus STIM2 (NP_{—}001099220.1), Mus musculus STIM2 (CAN36430.1), Bos taurus STIM2 (XP₋880249.3), Homo sapiens STIM2 (NP_{—}065911.2), Pan troglodytes STIM2 (XP_{—}001166811.1), Macaca mulatta STIM2 (XP_{—}001084422.1), Equus caballus STIM2 (XP_{—}001496930.2), Monodelphis domestica STIM2 (XP_{—}001366125.1), Gallus gallus STIM2 (XP_{—}420749.2), Danio rerio STIM2 (XP_{—}692356.3), Apis mellifera STIM1 (XP_{—}395207.3), Bombyx mori STIM1 (BAG68907.1), Drosophila melanogaster STIM (NP_{—}523357.2), Aedes aegypti STIM (XP_{—}001663818.1), Strongylocentrotus purpuratus STIM1 (XP_{—}001186683.1), Caenorhabditis elegans STIM1 (Q9N379_CAEEL).

FIG. 10. CAD-induced CRAC channel activity is Orai1-dependent and is sensitive to 2-APB and La³⁺. Ca²⁺ imaging of HEK 293 cells expressing Orai1 and CAD shows a sustained elevation of [Ca²⁺]ᵢ that is blocked by 100 μM 2-APB (A; n=19) or 10 μM La³⁺ (B; n=10). Data are shown as mean±sem. (C) Effect of 2-APB on constitutive I_{CRAC} in a HEK 293 cell expressing YFP-CAD+myc-Orai1. 50 μM 2-APB was added in the constant presence of 20 mM Ca²⁺ to the bath as indicated. 2-APB causes an increase in CAD-dependent current followed by a slower inhibition, identical to the typical response of I_{CRAC} when activated through store depletion. Current ramps a-c below show the current-voltage relation before, immediately after, and >100 s after application of 2-APB. (D) Inhibition of CAD-supported current by 10 μM LaCl₃. This YFP-CAD+myc-Orai1 transfected cell in 20 mM Ca²⁺ₒ was perfused with 20 mM Ca²⁺ₒ+10 μM LaCl₃ at the time indicated by bar, leading to rapid inhibition of current. Traces are not corrected for leak current.

FIG. 11. Cell surface trafficking and expression of Orai deletion constructs. (A) HEK 293 cells were transfected with either WT-Orai1 or the ΔN, ΔC or Δ73 Orai1 mutants containing an N-terminal YFP tag and an extracellular HA tag. Fifteen hours after transfection, the cells were fixed and stained with anti-HA antibodies prior to membrane permeabilization (left) or after permeabilization (right). In unpermeabilized cells, HA staining reflects cell surface Orai1 and YFP fluorescence reflects total expression of Orai1. (B) Quantification of cell surface Orai1 relative to the total expression of Orai1 in single cells (HA/YFP ratio) from the experiments shown in A. Data are shown as mean±sem (number of cells indicated). (C) Western blot of HEK 293 cells transfected with Flag-myc-CAD and GFP-Orai-DC, GFP-Orai1-DN or GFP-Orai1-DN73 for 16 h. Anti-GFP antibodies show that GFP-Orai1-DC is expressed at slightly higher levels than DN or DN73 Orai1 (upper panel; n=3). Staining with anti-Myc antibodies (lower panel; n=3) shows that the CAD polypeptide is well expressed under all conditions. Small differences in the levels of CAD polypeptide in different conditions were not consistently observed (n=3).

FIG. 12. Deletion of aa 73-84 from Orai1 suppresses CAD-induced Ca²⁺ influx. Ca²⁺ imaging in HEK 293 cells expressing CAD and wild type Orai1 (n=9), Orai1-ΔN (n=15), Orai1-ΔC (n=22), Orai1-Δ73-84 (n=14; eliminates CAD binding to the N-terminus), or Orai1_{L273S} (n=17; prevents CAD binding to the C-terminus). All of the mutations eliminate CAD binding sites and prevent activation of Orai1. All data presented as mean values±sem.

FIG. 13. Covariance method for identifying puncta containing STIM1 and Orai1. (A) Confocal images of cells expressing mCh-STIM1 and eGFP-Orai1, before and after store depletion (from FIG. 10). Pixels with covariance above threshold are used to generate binary masks which indicate the location of puncta where STIM1 and Orai1 colocalize. (B) Histograms of pixel covariance from the images in A before (Rest) and after (0 Ca+TG) store depletion. Each histogram is compiled from the covariance of pixels for which Sᵢ>s and Oᵢ>o, where Sᵢ and Oᵢ are the mCherry and eGFP fluorescence of pixel i, and s and o are the respective mean cell fluorescence values. The covariance threshold (dashed line) is set to two standard deviations above the mean covariance of the resting cell. (C) Example of the covariance method applied to STIM1₁₋₄₄₀, in which a larger fraction of STIM1 remains in the bulk ER after store depletion. Puncta are identified accurately even against a higher background of diffuse ER fluorescence. Confocal images are from FIG. 7C.

FIG. 14. Relation of Ca²⁺ influx rates to the expression of STIM1 variants. For each cell whose Ca²⁺ response was measured in FIG. 7E, expression of the mCherry-labeled STIM1 construct was quantified by normalizing its fluorescence to that of a bath-applied standard rhodamine solution. This value is plotted for each cell against the Ca²⁺ influx rate expressed as dR/dt measured 10-20 s after readdition of Ca²⁺ at 1100 s, where R is the fura-2 emission ratio with 350 and 380 nm excitation. Ca²⁺ influx is low or absent in STIM1-DCAD, STIM1₁₋₄₄₀, and STIM1 C437G despite expression levels similar to or greater than WT-STIM1. Relative GFP-Orai1 expression (mean±sem) was also measured in cells coexpressing each STIM1 variant, from the single-cell GFP fluorescence normalized to the fluorescence of a standard fluorescein solution: 3.47±0.34 (with STIM1, n=10), 2.41±0.23 (with STIM1-DCAD, n=9), 3.87±0.40 (with STIM1₁₋₄₄₈, n=12), 3.75±0.44 (with STIM1₁₋₄₄₀, n=10), and 3.84±0.26 (with STIM1 C437G, n=10). These data show that the lack of Ca²⁺ influx in cells transfected with STIM1-DCAD, STIM1₁₋₄₄₀, and STIM1 C437G are not due to abberrant expression of the STIM1 variant or Orai1.

FIG. 15. CAD (342-440) interacts with Orai1. YFP-Orai1 co-immunoprecipitates with Flag-myc-CAD (342-440) coexpressed in HEK 293 cells. Representative of 4 experiments.

### DEFINITIONS AND ABBREVIATIONS

The term “EF hand”, as used herein, relates to a helix-loop-helix structural domain found in a large family of calcium-binding proteins. It consists of two alpha helices positioned roughly perpendicular to one another and linked by a short loop region (usually about 12 amino acids) that usually binds calcium ions. The motif takes its name from traditional nomenclature used in describing the protein parvalbumin, which contains three such motifs and is probably involved in muscle relaxation via its calcium-binding activity. EF hands also appear in each structural domain of the signaling protein calmodulin and in the muscle protein troponin-C.

A “STIM polypeptide” or “STIM protein” is a polypeptide or protein that is the same as, a splice-variant of, or homologous to a naturally occurring STIM polypeptide or protein, or that is derived from such a polypeptide or protein (e.g., through cloning, recombination, mutation, or the like). The polypeptide can be full length or can be a fragment of a full length protein. A STIM fragment typically includes at least 10 contiguous amino acids corresponding to a native STIM protein. The polypeptide or protein can be naturally occurring or recombinant, and can be unpurified, purified, or isolated, and can exist, e.g., in vitro, in vivo, or in situ. The STIM polypeptide is a member of a highly conserved gene family that includes two known homologs (STIM1, STIM2) in the human STIM family of proteins. Any of a variety of STIM polypeptides or proteins and coding nucleic acids can be used in the present invention.

The term “CAD”, as used herein, is an acronym for a 107-residue CRAC activation domain within the C-terminus of STIM1. The CAD sequence is (SEQ ID NO:1):

YAPEALQKWLQLTHEVEVQYYNIKKQNAEKQLLVAKEGAEKIKKKRNTL
FGTFHVAHSSSLDDVDHKILTAKQALSEVTAALRERLHRWQQIEILCGF
QIVNNPGIH.

The term “SOCs”, as used herein, denominates store-operated Ca²⁺ channels. The term “SOCE”, as used herein, is an acronym for store-operated Ca²⁺ entry. The term “I_{CRAC}”, as used herein, denominates Ca²⁺ release-activated Ca²⁺ current. The terms “intracellular calcium” and “intracellular Ca²⁺”, as used herein, generally refer to “cytosolic calcium” and “cytosolic Ca²⁺” in a cell. The terms “intracellular stores”, “Ca²⁺ stores and “calcium stores”, as used herein, generally refer to calcium that is sequestered in the endoplasmic reticulum or other organelles in a cell.

An “Orai polypeptide” or “Orai protein” is a polypeptide or protein that is the same as, a splice-variant of, or homologous to a naturally occurring Orai polypeptide or protein, or that is derived from such a polypeptide or protein (e.g., through cloning, recombination, mutation, or the like). The polypeptide can be full length or can be a fragment of a full length protein. An Orai fragment typically includes at least 10 contiguous amino acids corresponding to a native Orai protein. The polypeptide or protein can be naturally occurring or recombinant, and can be unpurified, purified, or isolated, and can exist, e.g., in vitro, in vivo, or in situ. The Orai polypeptide is a member of a highly conserved gene family that includes three known homologs (Orai 1, Orai2 and Orai 3) in the human Orai family of proteins. Any of a variety of Orai polypeptides or proteins and coding nucleic acids can be used in the present invention.

Inhibitory Orai polypeptides include:

### (SEQ ID NO:2) Orai NT (1-91)

### MHPEPAPPPSRSSPELPPSGGSTTSGSRRSRRRSGDGEPPGAPPPPPSAVTYPDWIGQS YSEVMSLNEHSMQALSWRKLYLSRAKLKASSR

### (SEQ ID NO:3) Orai NT (48-91)

### SAVTYPDWIGQSYSEVMSLNEHSMQALSWRKLYLSRAKLKASSR

### (SEQ ID NO:4) Orai NT (64-91)

### MSLNEHSMQALSWRKLYLSRAKLKASSR

### (SEQ ID NO:5) Orai II-III (142-177)

### TCILPNIEAVSNVHNLNSVKESPHERMHRHIELAWA

### (SEQ ID NO:6) Orai CT (256-301)

HFYRSLVSHKTDRQFQELNELAEFARLQDQLDHRGDHPLTPGSHYA. When such peptides are brought into contact with a CRAC channel they will inhibit the opening of the channel and ion influx there through, for example by inhibiting endogenous Stim proteins. Such inhibition can have the effect of inhibiting a T cell mediated immune response.

The term “CRAC channel”, as used herein, denominates the calcium release-activated calcium channel, which belongs to the group of store-operated channels.

The term “puncta”, as used herein, describes specific areas where the STIM1 and/or Orai1 accumulate after depletion of intracellular calcium stores. This occurs at ER-plasma membrane junctions where the endoplasmic reticulum comes in close proximity to the plasma membrane.

The term “recombinant” or “recombinantly”, as used herein, has the usual meaning in the art and refers to a polynucleotide synthesized or otherwise manipulated in vitro. When used with reference to a cell, the term indicates that the cell replicates a heterologous nucleic acid or expresses a peptide or protein encoded by such a heterologous nucleic acid.

The term “heterologous nucleic acid”, as used herein, describes a nucleic acid that originates from a source foreign to the particular host cell, or, if from the same source, that is modified from its original form.

Permeant Domain. A number of permeant domains are known in the art and may be used in the present invention, including peptides, peptidomimetics, and non-peptide carriers. In one embodiment, the permeant peptide is derived from the third alpha helix of Drosophila melanogaster transcription factor Antennapaedia, referred to as penetratin, which comprises the amino acid sequence RQIKIWFQNRRMKWKK. In another embodiment, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

A “modulator” is an agent that modulates an activity of a given polypeptide or protein, e.g., an Orai and/or Stim polypeptide or protein. The agent can be any compound, molecule, element, substance, entity, or a combination thereof. It includes, but is not limited to, e.g., polypeptides, oligopeptides, small organic molecules, polysaccharides, polynucleotides including polynucleotides that encode a gene product of interest and/or that can act as a cell modulator without transcription and/or without translation. The agent can also be a ligand or antibody that specifically binds an Orai and/or Stim polypeptide or protein. It can be a natural product, a synthetic or semi-synthetic product, a chemical compound, or a combination of two or more substances.

The term “modulate” with respect to such a polypeptide or protein refers to a change in an activity or property of the polypeptide or protein. For example, modulation can cause an increase or a decrease in polypeptide or protein activity, binding characteristic (e.g., binding between Orai and Stim), or any other biological, functional, or immunological property of such a polypeptide or protein. The change in activity can arise from, for example, an increase or decrease in expression of one or more genes that encode these polypeptides or proteins, the stability of an mRNA that encodes the polypeptide or protein, translation efficiency, or from a change in activity of the polypeptide or protein itself. For example, a molecule that binds to Orai and/or Stim can cause an increase or decrease in a biological activity of the polypeptide(s) or protein(s). The terms “protein(s)” and “polypeptide(s)” are used interchangeably.

The terms “contact”, “contacts”, “contacting” have their normal meaning and refer to combining two or more entities (e.g., two proteins, a polynucleotide and a cell, a cell and a candidate agent, etc.). Contacting can occur in vitro, in situ or in vivo and is used interchangeably with “expose to”, “exposed to”, “exposing to.”

As used herein, the terms “reduce”, “decrease” and “inhibit” are used together because it is recognized that, in some cases, an observed activity can be reduced below the level of detection of a particular assay. As such, it may not always be clear whether the activity is “reduced” or “decreased” below a level of detection of an assay, or is completely “inhibited”.

The term “purified”, as used herein, denotes proteins and polypeptides that are isolated or separated from other proteins, polypeptides or contaminants with which they are naturally associated and that make up at least 50% of the total protein content of the composition containing the protein. “Purified” and “isolated” are used interchangeable herein.

Purification of Orai and/or Stim, can be accomplished using known techniques.

Generally, when purification is desired, cells expressing Orai and/or Stim are lysed, possibly crude purified and, subsequently, purified by chromatography to the desired level of purity. Cells can be lysed by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

The term “YFP” is an acronym for yellow fluorescent protein. The term “GFP” is an acronym for green fluorescent protein. The term “CFP” is an acronym for cyan fluorescent protein. The term “NFAT” is an acronym for “nuclear factor of activated T-cells (NFAT)”, which encompasses a family of transcription factors shown to be important in T cell-mediated immune response. The term “CAD-like proteins or polypeptides” denominates proteins or polypeptides that share a sequence homology of at least 50% with the CAD protein. The term “Orai-derived proteins or polypeptides” denominates proteins or polypeptides that share a sequence homology of at least 50% with the Orai protein.

### DETAILED DESCRIPTION

Methods and compositions are provided for modulating the calcium influx mediated by CRAC channels, and in particular modulating calcium influx in T lymphocytes, thereby altering immune responses mediated by such T lymphocytes. In some embodiments of the invention, a CRAC channel is contacted with a polypeptide that inhibits opening of the CRAC channel. In other embodiments of the invention, a CRAC channel is contacted with a polypeptide that enhances opening of the CRAC channel. These inhibiting or enhancing polypeptides may be referred to as modulating polypeptides.

Polypeptides are provided that comprise, consist or consist essentially of a minimal, highly conserved 107-amino acid residue STIM1 domain that binds to Orai1 and opens the CRAC channel, as well as the Orai peptides: Orai NT amino acids 1-91; Orai NT amino acids 48-91; Orai NT amino acids 64-91; Orai II-III amino acids 142-177; and Orai CT amino acids 256-301. The polypeptide and fragments and derivatives thereof provide for a variety of cellular processes, including T-lymphocyte activation and adaptive immunity, mast cell degranulation, platelet activation, and muscle development.

Embodiments of the present invention also include compositions and methods for investigating the activation process of the CRAC channel. Further embodiments include compositions and methods to design, identify and evaluate agents that modulate calcium influx in cells that contain store-operated channels, including T-lymphocytes, mast cells, and platelets as a treatment option for cases of immunological disorders, compromised immune function, organ transplantation, or thrombosis.

Identified herein is a minimal, highly conserved 107-amino acid residue CRAC activation domain (CAD) within the C-terminus of STIM1 that binds directly to the N- and C-termini of Orai1 to open the CRAC channel and were so able to establish a molecular activation mechanism for store-operated Ca²⁺ entry in which the direct binding of STIM1 to Orai1 drives the accumulation and the activation of CRAC channels at ER-PM junctions.

Using a variety of assays with truncated versions of the STIM1 protein, it is shown herein that a truncated version of STIM1 binds directly and tightly to Orai1. Truncated STIM1 protein STIM1 342-448 defines the minimal region that is necessary and sufficient to active NFAT, and is herein termed CRAC activation domain, in short CAD.

One aspect of the present invention is a vector that contains truncated versions of the STIM1 protein or a cell that expresses a truncated version of the STIM1 protein, particularly the CAD polypeptide, or the Orai peptides described herein. Such nucleic acids can be introduced into cells in cloning and/or expression vectors to facilitate introduction of the nucleic acid and expression of Orai and/or STIM to produce Orai and/or STIM. Vectors include, e.g., plasmids, cosmids, viruses, YACs, bacteria, poly-lysine, etc. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) artificial chromosomes. “Expression vectors” are vectors that comprise elements that provide for or facilitate transcription of nucleic acids which are cloned into the vectors. Such elements can include, e.g., promoters and/or enhancers operably coupled to a nucleic acid of interest.

Appropriate expression vectors are known in the art. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as yeast cells, e.g., Pichia, and Aspergillus cells; insect cells such as Drosophila S2 and Spodoptera Sf9 cells, mammalian cells such as CHO, COS, and HeLa; and even plant cells.

For various purposes the polypeptides of the invention may be fused to, or expressed in combination with, a permeant domain and/or a detectable marker. Such markers are known in the art. A suitable marker can be expressed in a desired host cell and will readily provide a detectable signal that can be assessed qualitatively and/or quantitatively, and can be detected directly or indirectly. Exemplary markers include fluorescent polypeptides including, but not limited to, YFP, CFP, GFP, mCherry and the like, or variants thereof. Methods of detecting, monitoring and measuring fluorescence are well known in the art.

CAD, CAD-derived peptides and Orai-derived peptides that bind CAD or CAD-like proteins as therapeutic agents. CAD, CAD-derived peptides and CAD-binding peptides from Orai can be modified to cross cell membranes using several approaches, including fusing the peptides to Tat sequence, poly-arginine motifs, viral peptides, or chemical modifiers such as polyethylene glycol, to facilitate uptake across the plasma membrane of intact cells. Upon such modification, CAD, CAD-derived peptides and Orai-derived peptides that are capable of binding CAD or CAD-like peptides are developed as therapeutic agents for cases of immunological disorders, compromised immune function, organ transplantation, or thrombosis.

### Modulation of T Cell Activation

Increasing CRAC channel activation in T cells augments the output of T cell receptor signaling, as indicated, inter alia, by the elevation of intracellular calcium, cytokine production, and cell proliferation. Inhibiting CRAC channel activation inhibits T cell receptor signaling.

As indicated above, the CRAC modulatory agent can be introduced into the target cell(s) using any convenient protocol, where the protocol will vary depending on whether the target cells are in vitro or in vivo. A number of options can be utilized to deliver the agent into a cell or population of cells such as in a cell culture, tissue, organ or embryo. Various physical methods are generally utilized in such instances, such as contact with a polypeptide comprising a permeant domain, contact with an expression vector, administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the agent, liposome-mediated transfection, or transfection using chemicals such as calcium phosphate. A number of established gene therapy techniques can also be utilized to introduce the agent into a cell. By introducing a viral construct within a viral particle, for instance, one can achieve efficient introduction of an expression construct into the cell and transcription of the RNA encoded by the construct.

For example, the modulatory agent can be injected into the host organism containing the target gene. The agent may be directly introduced into the cell (i.e., intracellularly); or introduced extracellularly into a cavity, interstitial space, into the circulation of an organism, introduced orally, etc. Physical methods of introducing nucleic acids include injection directly into the cell or extracellular injection into the organism of a solution. A nucleic acid agent may be introduced in an amount which allows delivery of at least one copy per cell. Higher doses (e.g., at least 5, 10, 100, 500 or 1000 copies per cell) of the agent may yield more effective modulation; lower doses may also be useful for specific applications.

When liposomes are utilized, substrates that bind to a cell-surface membrane protein associated with endocytosis can be attached to the liposome to target the liposome to T cells and to facilitate uptake. Examples of proteins that can be attached include capsid proteins or fragments thereof that bind to T cells, antibodies that specifically bind to cell-surface proteins on T cells that undergo internalization in cycling and proteins that target intracellular localizations within T cells. Gene marking and gene therapy protocols are reviewed by Anderson et al. (1992) Science 256:808-813.

In certain embodiments, a hydrodynamic nucleic acid administration protocol is employed. Where the agent is a ribonucleic acid, the hydrodynamic ribonucleic acid administration protocol described in detail below is of particular interest. Where the agent is a deoxyribonucleic acid, the hydrodynamic deoxyribonucleic acid administration protocols described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349; are of interest.

Additional nucleic acid delivery protocols of interest include, but are not limited to: those described in U.S. patents of interest include 5,985,847 and 5,922,687 (the disclosures of which are herein incorporated by reference); WO/11092; Acsadi et al., New Biol. (1991) 3:71-81; Hickman et al., Hum. Gen. Ther. (1994) 5:1477-1483; and Wolff et al., Science (1990) 247: 1465-1468; etc.

Depending on the nature of the agent, the active agent(s) may be administered to the host using any convenient means capable of resulting in the desired modulation of CRAC in the target cell. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

Introduction of an effective amount of an agent into a mammalian cell as described above results in a modulation of T cell activation.

The above described methods work in any mammalian cell, where representative mammal cells of interest include, but are not limited to cells of: ungulates or hooved animals, e.g., cattle, goats, pigs, sheep, etc.; rodents, e.g., hamsters, mice, rats, etc.; lagomorphs, e.g., rabbits; primates, e.g., monkeys, baboons, humans, etc.; and the like.

Before, during, or after treatment, the host may be assessed for immune responsiveness to a candidate antigen by various methods known in the art. The diagnosis may determine the level of reactivity, e.g. based on the number of reactive T cells found in a sample, as compared to a negative control from a naive host, or standardized to a data curve obtained from one or more patients. In addition to detecting the qualitative and quantitative presence of reactive T cells, the T cells may be typed as to the expression of cytokines known to increase or suppress inflammatory responses. It may also be desirable to type the epitopic specificity of the reactive T cells.

T cells may be isolated from patient peripheral blood, lymph nodes, or preferably from the site inflammation. Reactivity assays may be performed on primary T cells, or the cells may be fused to generate hybridomas. Such reactive T cells may also be used for further analysis of disease progression, by monitoring their in situ location, T cell receptor utilization, etc. Assays for monitoring T cell responsiveness are known in the art, and include proliferation assays and cytokine release assays.

Proliferation assays measure the level of T cell proliferation in response to a specific antigen, and are widely used in the art. In an exemplary assay, patient lymph node, blood or spleen cells are obtained. A suspension of from about 10⁴ to 10⁷ cells, usually from about 10⁵ to 10⁶ cells is prepared and washed, then cultured in the presence of a control antigen, and test antigens. The test antigens may be any peptides of interest. The cells are usually cultured for several days. Antigen-induced proliferation is assessed by the monitoring the synthesis of DNA by the cultures, e.g. incorporation of ³H-thymidine during the last 18H of culture.

Enzyme linked immunosorbent assay (ELISA) assays are used to determine the cytokine profile of reactive T cells, and may be used to monitor for the expression of such cytokines as IL-2, IL-4, IL-5, γIFN, etc. The capture antibodies may be any antibody specific for a cytokine of interest, where supernatants from the T cell proliferation assays, as described above, are conveniently used as a source of antigen. After blocking and washing, labeled detector antibodies are added, and the concentrations of protein present determined as a function of the label that is bound.

The peptides may be defined by screening with a panel of peptides derived from the test protein. The peptides will have at least about 8 and not more than about 30 amino acids, more usually not more than about 20 amino acids in length. A panel of peptides may represent the length of a protein sequence, i.e. all residues are present in at least one peptide.

Where the agent is acting to decrease T cell activation, the net effect is to increase the threshold for antigen signaling, and to decrease the sensitivity of a T cell to antigen, and conversely for an increase in T cell activation. The effect may be mediated in mature T cells, e.g. non-naïve T cells that have been exposed to an antigen of interest. Alternatively the target cell may be a progenitor to such mature T cells. Conditions of interest for downregulating T cells responses include allergic responses, autoimmune diseases, and in conjunction with transplantation, where graft rejection may occur as a result of T cell mediated immune responses.

Immune related diseases include: autoimmune diseases in which the immune response aberrantly attacks self-antigens, examples of which include but are not limited to multiple sclerosis (MS), acute disseminated encephalomyelitis (ADEM), rheumatoid arthritis (RA), type I autoimmune diabetes (IDDM), atherosclerosis, systemic lupus erythematosus (SLE), anti-phospholipid antibody syndrome, Guillain-Barre syndrome (GBS) and its subtypes acute inflammatory demyelinating polyradiculoneuropathy, and the autoimmune peripheral neuropathies; allergic diseases in which the immune system aberrantly attacks molecules such as pollen, dust mite antigens, bee venom, peanut oil and other foods, etc.; and tissue transplant rejection in which the immune system aberrantly attacks antigens expressed or contained within a grafted or transplanted tissue, such as blood, bone marrow cells, or solid organs including hearts, lungs, kidneys and livers; and the immune response against tumors. Samples are obtained from patients with clinical symptoms suggestive of an immune-related disease or with an increased likelihood for developing such a disease based on family history or genetic testing.

Other immune related diseases include allergy, or hypersensitivity, of the immune system, including delayed type hypersensitivity and asthma. Most cases of “atopic” or “allergic” asthma occur in subjects whom also exhibit immediate hypersensitivity responses to defined environmental allergens, and challenge of the airways of these subjects with such allergens can produce reversible airway obstruction. Both T cells and mast cells (and other FcRI+ cells) can have effector cell and immunoregulatory roles in these disorders.

NKT cells constitute a lymphocyte subpopulation that are abundant in the thymus, spleen, liver and bone marrow and are also present in the lung. They develop in the thymus from the CD4⁺CD8⁺ progenitor cells and circulate in the blood, have distinctive cytoplasmic granules, and can be functionally identified by their ability to kill certain lymphoid tumor cell lines in vitro without the need for prior immunization or activation. The mechanism of NKT cell killing is the same as that used by the cytotoxic T cells generated in an adaptive immune response; cytotoxic granules are released onto the surface of the bound target cell, and the effector proteins they contain penetrate the cell membrane and induce programmed cell death. There is evidence that suggests NKT cells are involved in the pathogenesis of conditions including asthma and certain autoimmune diseases.

Where a patient is undergoing transplantation, it may be desirable to down-regulate generally or specifically the patient immune response. In such cases, the therapeutic agent may be introduced prior to, concurrently with, or following the transplantation.

Where the agent is acting to increase expression CRAC activation, the net effect is to increase the sensitivity of a T cell to antigen. Conditions of interest for upregulating T cell responsiveness include conditions where there is an inadequate immune response, e.g. in the induction of immune responsiveness to cancer, to chronically infected cells, and the like.

Increased activation finds use in eliciting an immune response in an autologous, allogeneic or xenogeneic host. For example, where a tumor cell or a chronically infected cell expresses a protein, or over-expresses the protein relative to normal cells, a cytolytic immune response may be induced, where the tumor cell or infected cell is preferentially killed. The antigen for such purposes may be from the same or a different species. As used herein, the term antigen is intended to refer to a molecule capable of eliciting an immune response in a mammalian host, which may be a humoral immune response, i.e. characterized by the production of antigen-specific antibodies, or a cytotoxic immune response, i.e. characterized by the production of antigen specific cytotoxic T lymphocytes. The agent is administered in combination with the tumor antigen.

Several methods exist which can be used to induce an immune response against weakly antigenic protein, i.e. autologous proteins, etc. The immunogen is usually delivered in vivo to elicit a response, but in some cases it is advantageous to prime antigen presenting cells, e.g. dendritic cells, ex vivo prior to introducing them into the host animal.

In one embodiment, polypeptide antigens are mixed with an adjuvant that will augment specific immune responses to the antigen, wherein the adjuvant comprises an agent that increases CRAC activity in the targeted cell. Vaccine antigens may be presented using microspheres, liposomes, may be produced using an immunostimulating complex (ISCOM), as is known in the art.

### Screening Assays

Compound screening identifies agents that modulate that modulate calcium influx in cells that contain store-operated channels, including T-lymphocytes, mast cells, and platelets as a treatment option for cases of immunological disorders, compromised immune function, organ transplantation, or thrombosis. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the interaction between STIM1 and Orai1. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to one or both of the proteins. The binding assays usually involve contacting a protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The protein utilized in such assays can be naturally expressed, cloned or synthesized.

Compounds that are initially identified by any of the foregoing screening methods can be further tested to validate the apparent activity. The basic format of such methods involves administering a lead compound identified during an initial screen to an animal that serves as a model for humans and then determining if calcium is in fact differentially regulated. The animal models utilized in validation studies generally are mammals. Specific examples of suitable animals include, but are not limited to, primates, mice, and rats.

Active test agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate gene product activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Compounds identified by the screening methods described above and analogs thereof, as well as polypeptides of the invention can serve as the active ingredient in pharmaceutical compositions formulated for the treatment of various disorders, particularly regulation of immune cells such as T cells. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, and intrathecal methods.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Fluorescence resonance energy transfer (FRET) is a method of detection that is used in some embodiments and that is well know in the art. It describes energy transfer between two chromophores, where one chromophore acts as a energy donor, while the other chromophore acts as an energy acceptor. Alternatively, binding of immunodetectable domains can be accomplished using a secondary antibody, that is detectably labeled, against an anti-immunodetectable domain antibody.

In further embodiments, [Ca²⁺]ᵢ was measured in HEK 293 cells expressing CAD and Orai1 and whole-cell patch clamp recordings were conducted from HEK 293 cells transiently transfected with myc-Orai1 and either YFP-STIM1 or YFP-CAD.

In further embodiments, YFP-CAD was expressed with or without myc-Orai1 in HEK 293 cells and its intracellular localization examined. In the absence of Orai1, YFP-CAD was localized diffusely throughout the cytoplasm, but the introduction of Orai1 led to a dramatic recruitment of YFP-CAD to the plasma membrane, suggesting that the two proteins form a complex.

In further embodiments, Flag-tagged CAD and GFP-tagged Orai1 were expressed in HEK 293T cells and CAD immunoprecipitated with anti-Flag antibodies.

In other embodiments, CAD and Orai1 were introduced into a yeast split ubiquitin interaction system (Thaminy et al., 2004) to verify the association of CAD and Orai1 in vivo, and to map the interaction domains of Orai1.

In further embodiments, a GST-tagged CAD was generated in E. coli and an Orai1 protein containing C-terminal octa-histidine and N-terminal EEYMPME (“EE”) tags was generated in insect Hi5 cells to test for direct binding between CAD and Orai1.

CAD's central role as the domain of the STIM1 protein that activates the CRAC channel is further underscored by the ability of CAD polypeptide or STIM1₁₋₄₄₈ to activate store-operated Ca²⁺ entry (SOCE) and by the loss of STIM1 activity following deletion of CAD or the introduction of mutations that inhibit CAD function.

In some embodiments, the subject methods are useful for identifying an endogenous gene product that has an activity in modulating the interaction between STIM and Orai proteins. Genes that have a beneficial effect on the phenotype when their activity is modulated through mutation encode proteins that represent therapeutic targets for the development of compound. Gene based therapies can be identified by doing traditional enhancer/suppressor analyses in the subject cells. In these analyses, genes in the subject cells are mutated to identify ones that either increase or decrease basal calcium levels.

Methods of mutating genes and carrying out enhancer/inhibitor analyses are well known to those of skill in the art (Hays, T S et al., Molecular and Cellular Biology (March 1989) 9(3):875-84; Deuring, R; Robertson, B; Prout, M; and Fuller, M T. Mol. Cell. Biol., 1989 9:875-84; Fuller, M T et al., Cell Mot. Cyto. (1989) 14:128-35; Rottgen G, Wagner T, Hinz U Mol. Gen. Genet. 1998 257:442-51). In some embodiments, siRNA might be used to disrupt the expression of an endogenous gene to determine whether the endogenous gene had an effect on modulating the interaction between STIM and Orai proteins.

One screening method involves 1) label the CAD or CAD-like protein with a fluorescent tag (e.g., YFP or GFP); 2) label Orai or Orai polypeptide with an orthogonal fluorescent tag (e.g., CFP or mCherry); 3) express the labeled CAD or CAD-like protein together with the Orai or Orai polypeptide in a cell transiently or stably; 4) contact the cell with a candidate agent for an appropriate time; 5) assess the effect of the candidate agent on localization of the CAD or CAD-like protein in the cell by fluorescence or total internal reflection fluorescence microscopy. 6) If the agent reduces the amount of CAD or CAD-like protein at the cell membrane, then the candidate agent is a potential inhibitor of STIM-Orai interaction and may also inhibit Ca²⁺ entry through store-operated channels. 7) If the agent increases the amount of CAD or CAD-like protein at the cell membrane, then the candidate agent is a potential enhancer of STIM-Orai interactions and may also activate Ca²⁺ entry through store-operated channels.

Another screening method using CAD or CAD-like proteins in vitro, is as follows: 1) Produce CAD, CAD-like protein, Orai and Orai-derived polypeptides in a cell expression system (e.g., E. coli, yeast, insect cells, mammalian cells) or synthesize the polypeptides chemically; 2) coat an ELISA plate with CAD, CAD-like, Orai or Orai-derived polypeptides; 3) introduce the complementary polypeptide (CAD if Orai is plated, or Orai if CAD is plated) labeled with a fluorescent or enzymatic tag, along with a candidate agent; 4) measure the concentration of CAD or Orai bound to its complement on the solid substrate using a fluorescent or enymatic readout 5) If the agent reduces the amount of labeled CAD or CAD-like protein (or of labeled Orai or Orai-derived polypeptides) bound to the solid substrate, then the candidate agent is a potential inhibitor of STIM-Orai interactions and may also inhibit Ca²⁺ entry through store-operated channels. 6) If the agent increases the amount of CAD or CAD-like protein bound to the solid substrate, then the candidate agent is a potential enhancer of STIM-Orai interactions and may also activate Ca²⁺ entry through store-operated channels.

Another screening method involves 1) Produce CAD, CAD-like protein, Orai or Orai-derived polypeptides in a cell expression system (e.g., E. coli, yeast, insect cells, mammalian cells) or synthesize the polypeptides chemically; 2) label the CAD, CAD-like protein, Orai or Orai-derived polypeptides with a fluorescent protein or a reporter enzyme; 3) screen a compound library for binding to CAD, CAD-like protein, Orai or Orai-derived polypeptides using a variety of approaches, including solid substrate microarray or polystyrene beads, fluorescence polarization, and surface plasmon resonance. 4) compounds that bind to CAD, CAD-like protein, Orai or Orai-derived polypeptides are potential modulators of STIM-Orai interactions and may also modulate Ca²⁺ entry through store-operated channels.

Another screening methods involves 1) Produce CAD or CAD-like protein in E. coli or yeast or synthesize the polypeptides chemically; 2) label the CAD or CAD-like protein with a fluorescent protein or a reporter enzyme; 3) coat an ELISA plate with unlabeled CAD or CAD-like protein; 4) introduce the labeled CAD or CAD-like protein along with a candidate agent; 5) measure the concentration of labeled CAD or CAD-like protein bound to the solid substrate using a fluorescent or enzymatic readout 6) If the agent reduces the amount of CAD or CAD-like protein bound to the solid substrate, then the candidate agent is a potential inhibitor of STIM oligomerization and may also inhibit Ca²⁺ entry through store-operated channels. 7) If the agent increases the amount of CAD or CAD-like protein bound to the solid substrate, then the candidate agent is a potential enhancer of STIM oligomerization and may also activate Ca²⁺ entry through store-operated channels.

Another screening method involves 1) label the CAD or CAD-like protein with a fluorescent tag (e.g., YFP or GFP); 2) label Orai or Orai polypeptide with an orthogonal fluorescent tag that can act as a Fluorescence resonance energy transfer acceptor for the first; 3) express the labeled CAD or CAD-like protein together with the Orai or Orai polypeptide in a cell transiently or produce the two peptides in vitro; 4) contact the cell with a candidate agent for an appropriate time; 5) measure the amount of fluorescence energy transfer (FRET) between CAD and Orai peptides using a microscope, or other fluorescence detector. 6) If the agent reduces the amount of FRET between CAD and Orai, then the candidate agent is a potential inhibitor of STIM-Orai interaction and may also inhibit Ca²⁺ entry through store-operated channels. 7) If the agent increases the amount of FRET between CAD or Orai, then the candidate agent is a potential enhancer of STIM-Orai interactions and may also activate Ca²⁺ entry through store-operated channels.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are herein described.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### EXPERIMENTAL

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

Cells and Transfection. HEK 293 and HEK 293T cells (ATCC) were cultured in DMEM with GlutaMax (GIBCO, Carlsbad, Calif.), 10% FBS (Hyclone, Logan, Utah), and 1% penicillin/streptomycin (Mediatech, Hargrave, Va.). A HEK 293 cell line with an inducible eGFP-myc-Orai1 was generated using the Flp-In T-REx system (Invitrogen) and was maintained with 50 μg/ml hygromycin and 15 μg/ml blasticidin. Cells were transfected at 90% confluency with 0.2-0.5 μg DNA using Lipofectamine 2000 (Invitrogen) according to the manufacturer's instructions.

NFAT-luciferase Assays. HEK 293T cells were co-transfected with the indicated constructs and an NFAT reporter gene (firefly luciferase gene C-terminal to 4×-NFAT site from the IL-2 gene). Cotransfection with the Renilla luciferase (pRLTK) gene driven by the TK promoter was used to control for cell number and transfection efficiency. After 12-18 h, cells were treated with a control DMSO solution (mock), PMA (1 μM), or PMA plus TG (1 μM) for 8 h. Assays were performed using the Dual Luciferase Reporter assay system (Promega). For each condition, luciferase activity was measured with 4 samples taken from duplicate wells using a 96-well automated luminometer (Turner Biosystems). Results are represented as the ratio of firefly to Renilla luciferase activity.

Immunoprecipitation and Immunoblot Analysis. Transfected HEK 293T cells (12-24 h) were washed with PBS and lysed in 50 mM Tris-HCl pH 7.5, 150 mM NaCl, 1% Triton X-100, and protease inhibitors. Lysates were spun at 12,000 rpm for 10 min, and the supernatant was incubated with anti-Flag M2 agarose beads (Sigma), or anti-myc or -HA antibody followed by IgG agarose beads (Pierce). Lysates and immunoprecipitates were subjected to SDS-PAGE, probed with HRP-conjugated secondary antibody and detected by enhanced chemiluminescence (Pierce).

Split-Ubiquitin Yeast Two Hybrid Assay. Screening was performed according to the manufacturer's instructions (Dualsystems Biotech). Transformed yeast were selected on media lacking Trp and Leu (-TL). Interaction was observed by cell growth on plates lacking Trp, Leu, and His (TLH) in the presence of 3-aminotriazole (3-AT). Protein interactions were also assessed by measuring lacZ activity using the chromogenic substrate X-gal (5-bromo-4-chloro-3-indolyl-β-galactopyranoside).

GST Pull-down Assays. For GST pull-down assays, either GST-CAD (0.16 μM) or GST (0.15 μM) was incubated with EE-Orai1-His8 (0.1 μM) for 1 h in buffer containing (in mM): 20 Tris-HCl (pH 8.0), 150 NaCl, 20 imidazole, and 1 DTT with protease inhibitors. After addition of glutathione sepharose the beads were centrifuged, washed with the above buffer, and the precipitated proteins were eluted with boiling SDS and analyzed by SDS-PAGE and Western blotting.

Purification of EE-Orai1-His8. EE-Orai1-His8 and His6-CAD were integrated into baculoviruses and expressed alone (Orai1) or together (Orai1+CAD) in Hi5 cells (Invitrogen, Carlsbad, Calif.) for 48 h at 28° C. After cell lysis, membranes were solubilized by resuspension in 1% DDM (n-dodecyl-β-d-maltoside; Anatrace, Ill., USA). Protein was purified using Ni-NTA beads, and the eluted protein was then incubated with γ-bind Protein G Sepharose beads and anti-EE antibody (Covance, Calif., USA) overnight at 4° C., and eluted with 1 mg/ml EE polypeptide (Anaspec, Calif., USA). DDM was maintained at 0.1%, and the NaCl concentration was 0.5 M up to this purification step. The protein was then passed over a Superose 6 size exclusion column equilibrated in 20 mM Tris pH 8, 150 mM NaCl, 10% glycerol, 0.02% DDM and 0.004% CHS to remove aggregated material and EE peptide. Orai1 was >98% pure as judged by SDS-PAGE analysis.

Multi-angle Light Scattering (MALS) Analysis. For MALS analysis, the proteins purified after the Ni-NTA affinity step were concentrated using a 30 kDa cutoff vivaspin membrane and loaded on a Superose 6 column. Excess CAD eluted as a separate homogenous peak after the Orai1-CAD complex elution. A DAWN EOS light scattering system (Wyatt Technology, Santa Barbara, Calif.) equipped with a K5 flow cell, a 30-mW linearly polarized GaAs 690-nm laser, a pump (Model PU-980, Jasco Corp., Tokyo, Japan), and an HPLC Shodex KW-803 size exclusion column was used for determining the molar mass of CAD. The protein sample in running buffer (20 mM Tris pH 8, 150 mM NaCl, 10% glycerol, 0.02% DDM, 2 mM β-ME) was filtered through a 0.1 μm filter. 150 μg CAD was loaded at a concentration of 1 mg/ml. Both the light scattering unit and the refractometer were calibrated according to the manufacturer's instructions. A do/dc value of 0.185 ml/g was used. Light scattering data was used from 11 detectors ranging from 50° to 134° (detectors 6 through 16). The detector responses were normalized against monomeric bovine serum albumin.

MALS Analysis of His₆-CAD. His6-CAD was isolated from cells expressing His6-CAD and EE-Orai1-His8 and analyzed by MALS using a DAWN EOS light scattering system (Wyatt Technology, Santa Barbara, Calif.). The detector responses were normalized against monomeric bovine serum albumin.

Electron Microscopy. EE-Orai1-His8 alone or copurified with His6-CAD was diluted to a final concentration of ˜0.01 mg/ml in 20 mM Tris, 150 mM NaCl, and 0.02% DDM buffer and was negatively stained with uranyl formate as described (Ohi et al., 2004). Images were recorded with a Phillips CM-10 electron microscope equipped with a tungsten filament operated at 100 kV. Images were taken at a nominal magnification of 39,000× and a defocus of 1.5 μm on a Gatan 1 k×1 k CCD camera.

Confocal Microscopy. 6 h after transfection, HEK 293 cells were plated onto sterilized coverslips coated with poly-D-lysine and maintained in complete DMEM for an additional 12-18 h before imaging in Ringer's solution containing (in mM): 155 NaCl, 4.5 KCl, 2 CaCl2, 1 MgCl2, 10 D-glucose, and 5 Na-Hepes, pH 7.4. To deplete stores, cells were treated with 1 μM TG in Ca2+-free Ringer's (prepared by substituting 2 mM MgCl2 and 1 mM EGTA for CaCl2) for 10 min. eGFP and mCherry were excited simultaneously at 488 and 594 nm, respectively, on a Leica SP2 AOBS inverted confocal microscope equipped with a PL APO 100×/NA 1.4 oil immersion objective. Fluorescence emissions were collected at 615-840 nm (mCherry) and 510-570 nm (eGFP). All experiments were performed at 22-25° C.

Fluorescence Recovery After Photobleaching (FRAP). Inducible HEK 293 cells were transiently transfected with mCh-CAD and maintained with 10 μM LaCl3 to suppress chronic Ca2+ entry. After 15-16 h, eGFP-myc-Orai1 expression was induced with 1 μg/ml tetracycline in medium containing LaCl3. Cells were rinsed with 2 mM Ca2+ Ringer's solution at 24 h post-transfection prior to imaging on the confocal system described above. Prebleach and recovery images were scanned at 488 nm at low power (20 mW Ar laser, 50% power, 6% transmission). A 3-μm strip was bleached at 50% laser power with full transmission for ˜2 s. Bleaching during the recovery period was negligible (<5%). FRAP recovery curves were analyzed with ImageJ (W. S. Rasband, NIH, Bethesda, Md., following a published method in which an empirical formula approximating the one-dimensional solution of the diffusion equation is fitted to the time course of fluorescence recovery within the bleached ROI. An offset was introduced to account for incomplete bleaching. The

$$I ( t ) = {I}_{b} + ( {I}_{f} - {I}_{b} ) ( 1 - \sqrt{\frac{{w}^{2}}{{w}^{2} + 4 π Dt}} ) ,$$

where I(t) is the mean intensity within the bleached region at time t, I_{b} is the intensity immediately after bleaching, I_{f} is the intensity after full recovery, w is the width of the bleached region, and D is the effective diffusion coefficient, was fit to the FRAP recovery curves with Igor Pro 5.0 (Wavemetrics) to determine D. Mobility was determined as (I_{f}−I_{b})/(I₀−I_{b}), where I_{f} and I_{b} are defined as above, and I₀ is the initial intensity within the ROI after correction for the loss of whole-cell fluorescence by the bleaching pulse (˜10%).

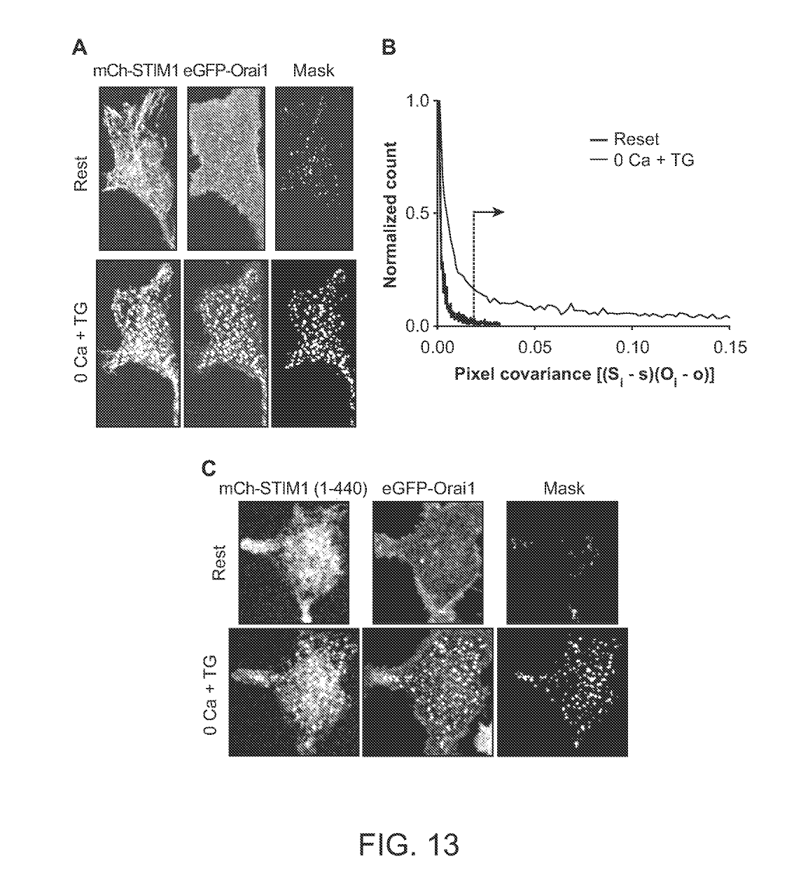

Quantitation of STIM1 and Orai1 Recruitment into Puncta. Colocalization of eGFP-Orai1 and mCh-STIM1 variants was analyzed from background-corrected confocal image pairs acquired before and after store depletion. Puncta have two defining characteristics: (1) because they are sites of accumulation, STIM1 and Orai1 fluorescence intensities in puncta are greater than the respective mean intensities measured across the entire cell, and (2) because STIM1 and Orai1 co-localize in puncta, their fluorescence covariance is high. For each STIM1/Orai1 image pair, we computed the covariance of each pixel as (Sᵢ−s) (Oᵢ−o), where Sᵢ and Oᵢ are the STIM1 and Orai1 fluorescence of pixel i, and s and o are the mean STIM1 and Orai1 fluorescence of the entire cell. A threshold criterion for puncta was set using the pre-depletion image pair. The mean and s.d. of the covariance values were calculated from pixels for which both Sᵢ>s and Oᵢ>o, and a covariance threshold was set equal to the mean+2×s.d. of this pre-depletion condition. Binary masks were applied to the pre- and post-depletion image pairs to isolate pixels for which Sᵢ>s and Oᵢ>o and whose covariance exceeds the pre-depletion threshold. Examples of the method are illustrated FIG. 13. Integrated STIM1 or Orai1 puncta intensity was calculated from the masked images and expressed as a fraction of the total fluorescence intensity in the unmasked image. Image analysis was carried out with ImageJ and the MacBiophotonics Intensity Correlation Analysis plug-in.

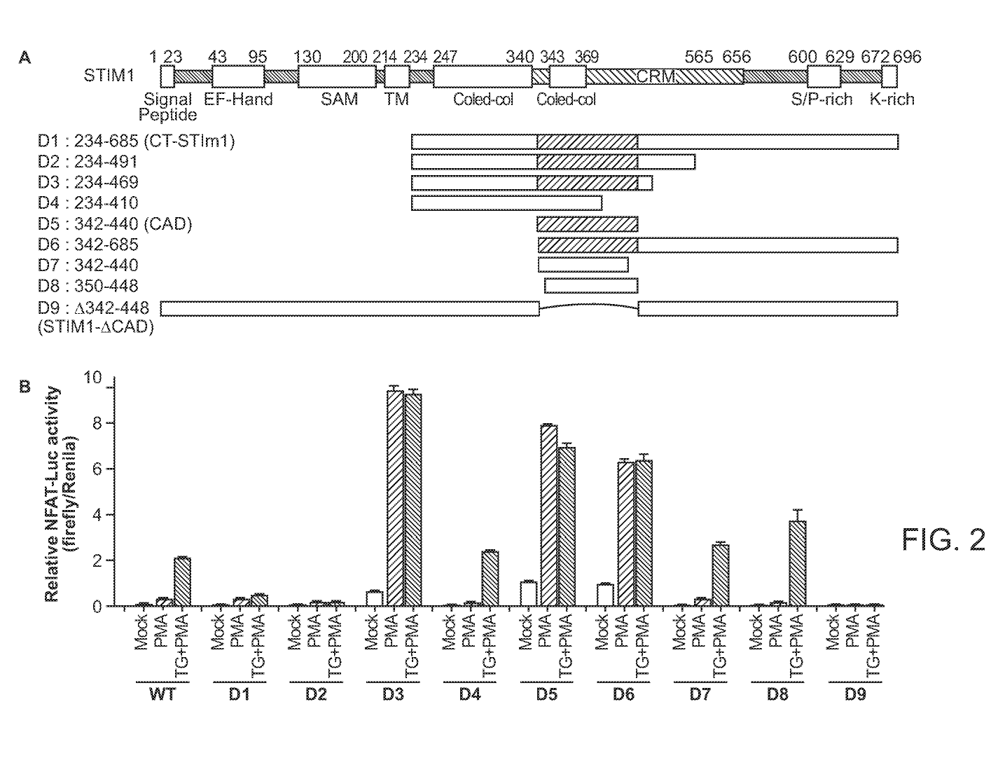

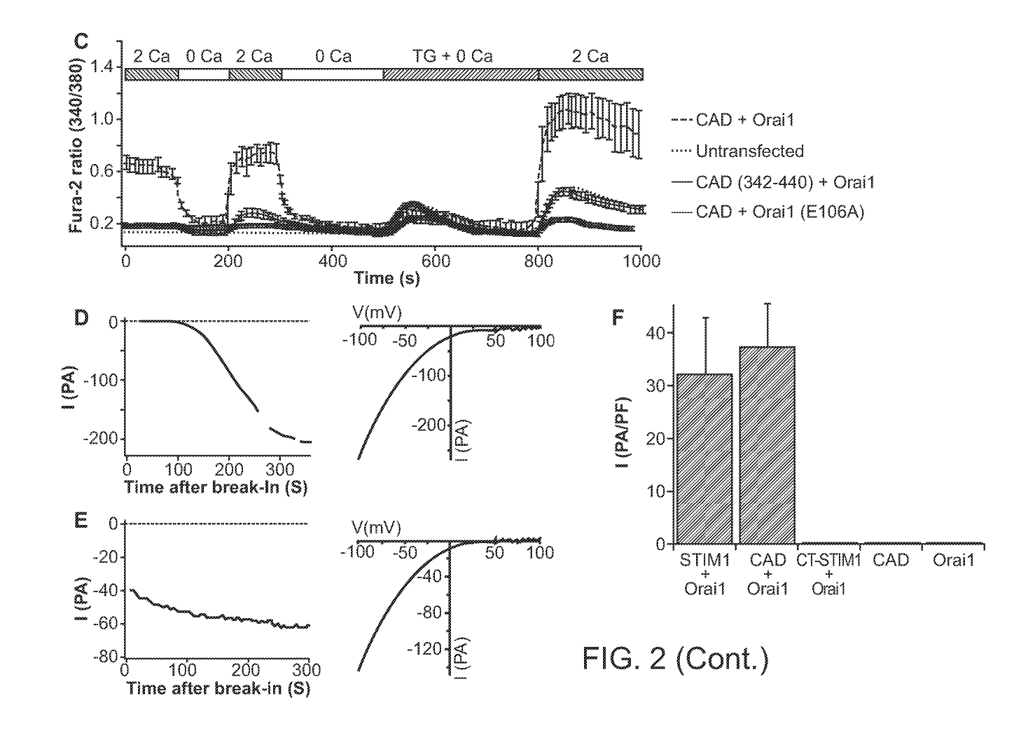

Ca²⁺ Imaging. For FIG. 2, cells were loaded at 37° C. in DMEM with 1 μM fura-2/AM for 30 min. Ratiometric Ca2+ imaging was performed at 340 and 380 nm in 2 mM Ca2+ Ringer's solution with a Nikon Eclipse 2000-U inverted microscope equipped with a fluorescent arc lamp, excitation filter wheel, and a Hamamatsu Orca CCD camera. Images were collected using Openlab (Improvision) and analyzed using Igor Pro.

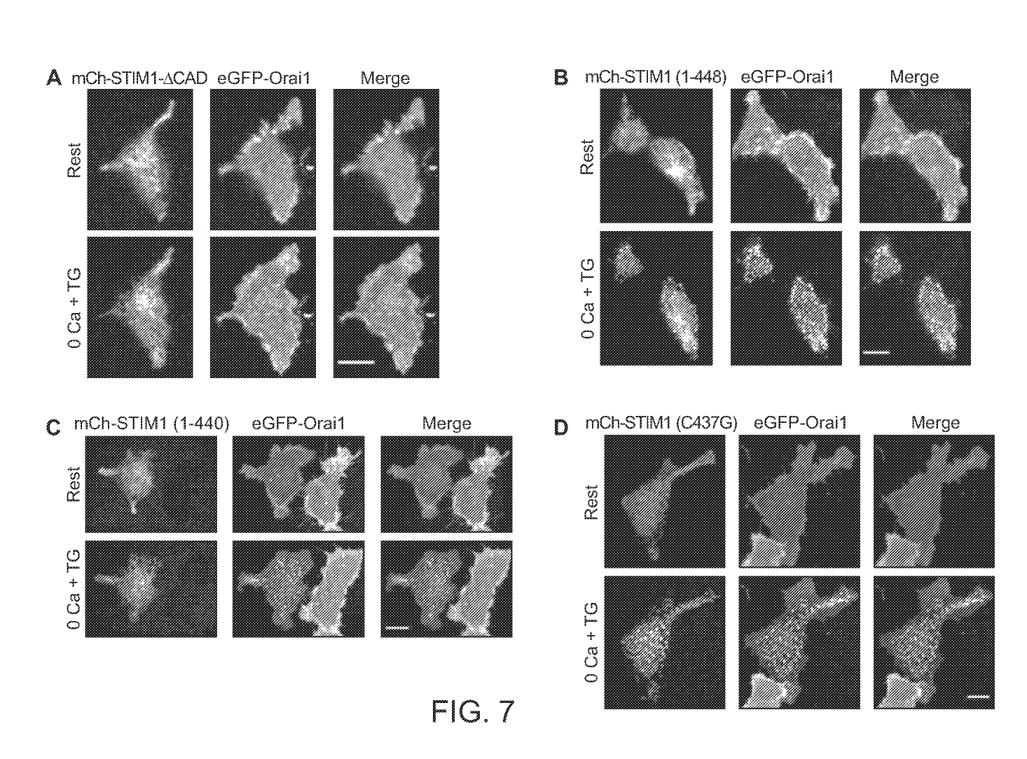

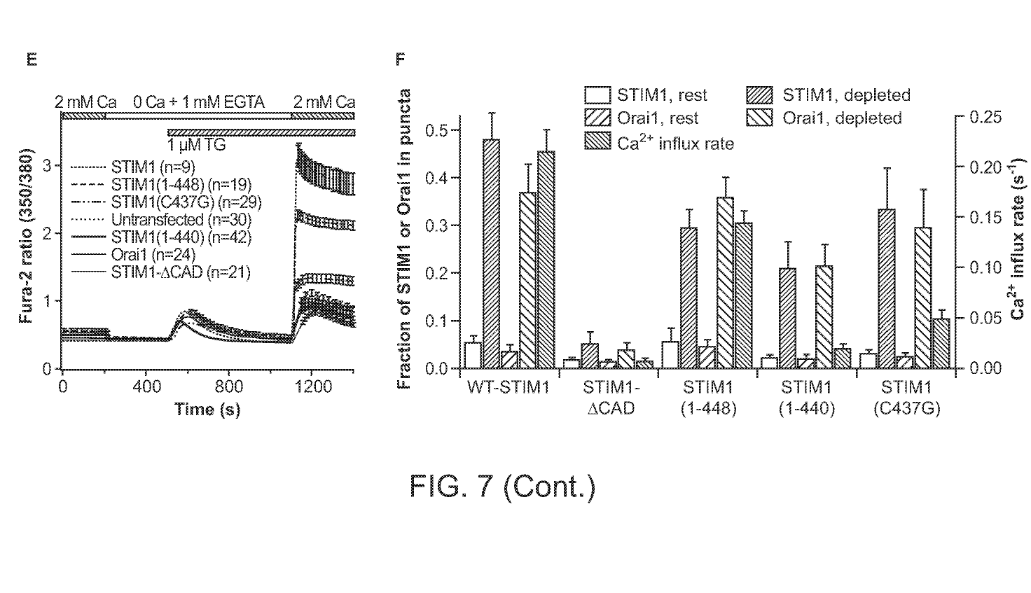

For FIG. 7, cells were loaded as above with 2 μM fura-2/AM for 25 min. Ratiometric Ca2+ imaging was performed with 350 and 380 nm excitation in 2 mM Ca2+ Ringer's solution on an Axiovert 35 inverted microscope using a VideoProbe imaging system as described (Bautista et al., 2002). mCherry-positive cells were identified using a 540±12 nm excitation and a 580LP emission filter (Chroma).

Electrophysiology. HEK 293 cells were transfected 8-24 h prior to electrophysiology experiments with STIM1- and Orai1-derived constructs in a 1:1 mass ratio using Lipofectamine 2000. Cells transfected with CAD+Orai1 were cultured in 10 μM LaCl3 to avoid the toxicity of constitutively active I_{CRAC}, and LaCl3 was washed out immediately before seal formation. ICRAC in cells cultured without LaCl3 was similar to that in cells cultured with LaCl3, but most cells without LaCl3 died soon after break-in.

Currents were recorded using standard whole-cell patch clamp techniques (Prakriya and Lewis, 2001). Pipettes of resistance 2-5 MΩ were filled with an internal solution containing (in mM) 150 Cs aspartate, 8 MgCl2, 10 EGTA, and 10 HEPES (pH 7.2 with CsOH). Currents were sampled at 5 kHz and filtered at 2 kHz, and all voltages were corrected for the junction potential of the pipette solution relative to Ringer's in the bath (−13 mV).

Patch-clamp Experiments. Pipette-membrane seals were formed in 2 mM Ca²⁺ Ringer's solution. After break-in to the whole-cell configuration, voltage stimuli consisting of a 100-ms step to −100 mV followed by a 100-ms ramp from −100 to +100 mV were delivered every 5 s from the holding potential of +30 mV. After the currents reached a steady level (˜300 s for wild-type STIM1), the external solution was changed via a multibarrel local perfusion pipette to a high-Ca²⁺ Ringer's containing (in mM) 130 NaCl, 4.5 KCl, 20 CaCl₂, 1 MgCl₂, 10 D-glucose, and 5 HEPES (pH 7.4 with NaOH). All data were leak-corrected using the current elicited in 20 mM Ca²⁺ Ringer's+10 μM LaCl₃ at the end of the each experiment. LaCl₃ and 2-APB were added directly to parent solutions on the day of the experiment from 10 mM and 100 mM stocks in H₂O and DMSO, respectively.

Measurement of Orai1 Cell Surface Expression. HEK 293 cells were cultured on 10 mm coverslips and transfected with the extracellular HA-tagged Orai1 constructs as indicated. 16 h after transfection, the cells were fixed with 4% paraformaldehyde, 8% sucrose in PBS for 10 min in the absence or presence of 0.25% Triton X-100. After 1 hr blocking with 3% BSA/PBS, cells were stained with anti-HA antibody (rat, 3F10; 1:1000 dilution in 3% BSA/PBS) for 2 hr at 4° C., washed 5 times and detected with a secondary goat anti-rat-Alexa 594 (1:1000 dilution, Molecular Probes). The ratio of YFP to HA-Alexa 594 fluorescence was measured in the whole cell using widefield epifluorescence microscopy. The cells were analyzed blind.

Materials and Antibodies. 2-Aminoethoxydiphenylborate (2-APB) and 3-amino-1,2,4-triazole (3-AT) were obtained from Sigma. Fura-2/AM, Lipofectamine 2000 and 5-bromo-4-chloro-3-indolyl-□-D-galactosidase (X-gal) were obtained from Invitrogen. Thapsigargin and phorbol 12-myristate 13-acetate (PMA) were from LC Laboratories, anti-Flag M2 affinity gel was obtained from Sigma, and Immunopure protein G beads were from Pierce. Antibodies targeting myc (4A6, Upstate), HA (3F10, Roche), GFP (598, MBL), GST (Santa Cruz Biotechnology), and 6×HIS (Qiagen) were purchased from the indicated vendor.

Plasmids. mCh-STIM1 and eGFP-myc-Orai1 plasmids were described previously (Luik et al., 2006). mCh-STIM1-DK was constructed by substituting nucleotides at positions 2011 (c→t) and 2013 (g→a) (Quickchange XL; Stratagene) with primer 5′-gactccagcccaggctgaaagaaglitcctctc-3′ to generate a premature stop codon. For comparison purposes, YFP-CT-STIM1 (236-685) was kindly provided by C. Romanin (University of Linz, Austria) and S. Muallem (236-685; K680Q mutation; UT Southwestern, Dallas, Tex.) and HA-CT-STIM1 (237-685) by M. Cahalan (UC Irvine, Calif.). Orai1-ΔN, Orai1-ΔC and Orai1-ΔN73 were kindly provided by T. Xu (Chinese Academy of Sciences, Beijing, China). STIM1 or Orai1 fragments were amplified by PCR using the primer sets shown below. The PCR products were cloned into the pCR8 TOPO-adapted cloning vector (Invitrogen). The expression constructs were generated by transferring the pCR8 PCR products into destination plasmids using Gateway technology (Invitrogen). Sequences of all constructs were verified.

For STIM1 cyto 234-685 (CT-STIM1): Forward (SEQ ID NO:1) AACCGTTACTCCAAGGAGCAC; Reverse (SEQ ID NO:2) GGAATTCCTACTTCTTAAGAGGCTTCTTAAAG; For STIM1 234-491: Forward (SEQ ID NO:3) AACCGTTACTCCAAGGAGCAC; Reverse (SEQ ID NO:4) TCACTGCATGGACAAGGGAGACAC; For STIM1 234-469: Forward (SEQ ID NO:5) AACCGTTACTCCAAGGAGCAC, Reverse (SEQ ID NO:6) TCAAGCAGGGTTGGGGCGTGTACTGCC. For STIM1 234-410: Forward (SEQ ID NO:7) AACCGTTACTCCAAGGAGCAC; Reverse (SEQ ID NO:8) GAATTCAATGATCTACATCATCCAGGGAAG. For STIM1 342-448 (CAD): Forward (SEQ ID NO:9) GGATCCATGTATGCTCCAGAGGCCCTTC, Reverse (SEQ ID NO:10) GAATTCAGTGGATGCCAGGGTTGTTG. For STIM1 342-685: Forward (SEQ ID NO:11) GGATCCATGTATGCTCCAGAGGCCCTTC; Reverse (SEQ ID NO:12) GGAATTCCTACTTCTTAAGAGGCTTCTTAAAG. For STIM1 342-440: Forward (SEQ ID NO:13) GGATCCATGTATGCTCCAGAGGCCCTTC; Reverse (SEQ ID NO:14) TCACTGGAAGCCACAGAGGATCTCGAT. For STIM1 350-448: Forward (SEQ ID NO:15) GGATCCATGTGGCTGCAGCTGACACATGAG Reverse (SEQ ID NO:16) GAATTCAGTGGATGCCAGGGTTGTTG. For STIM1 deltaCAD: Forward #1 (SEQ ID NO:17) CATGGTATGCTCCAGATATCCTTCAGAAGTGGCT; Reverse #1 (SEQ ID NO:18) AGCCACTTCTGAAGGATATCTGGAGCATACCATG; Forward #2 (SEQ ID NO:19) ATTGTCAACAACCCTGATATCCACTCACTGGTG; Reverse #2 (SEQ ID NO:20) CACCAGTGAGTGGATATCAGGGTTGTTGACAAT. For Orai1 1-91: Forward (SEQ ID NO:21) ATGCATCCGGAGCCCGCCCCGCCCCCG; Reverse (SEQ ID NO:22) TCACCGGCTGGAGGCTTTAAGCTT. For Orai1 1-70: Forward (SEQ ID NO:23) ATGCATCCGGAGCCCGCCCCGCCCCCG; Reverse (SEQ ID NO:24) TCAGGAGTGCTCGTTGAGGCTCAT. For Orai1 48-91: Forward (SEQ ID NO:25) GGATCCATGTCCGCCGTCACCTAC; Reverse (SEQ ID NO:26) TCACCGGCTGGAGGCTTTAAGCTT. For Orai1 48-70: Forward (SEQ ID NO:27) GGATCCATGTCCGCCGTCACCTAC; Reverse (SEQ ID NO:28) TCAGGAGTGCTCGTTGAGGCTCAT. For Orai1 68-91: Forward (SEQ ID NO:29) GGATCCATGGAGCACTCCATGCAGGCGCTG; Reverse (SEQ ID NO:30) TCACCGGCTGGAGGCTTTAAGCTT. For Orai1 142-177: Forward (SEQ ID NO:31) GGATCCACCTGCATCCTGCCCAACATCGAG; Reverse (SEQ ID NO:32) TCAGGCCCAGGCCAGCTCGATGTGGCG. For Orai1 255-301: Forward (SEQ ID NO:33) GGATCCGTCCACTTCTACCGCTCACTG; Reverse (SEQ ID NO:34) CTAGGCATAGTGGCTGCCGGGCGTCAG. For Orai1 E106A mutation: Forward (SEQ ID NO:35) GTGGCAATGGTGGCGGTGCAGCTGGAC; Reverse (SEQ ID NO:36) GTCCAGCTGCACCGCCACCATTGCCAC.

N-terminally myc-tagged WT Orai1 in the Gateway entry vector pENTR11 (a gift from S. Feske, Harvard Medical School, Boston, Mass.) was inserted into the custom-designed Gateway destination vector pDEST-pGWI by recombination reaction using enzyme mix (Gateway LR Clonase; Invitrogen, Carlsbad, Calif.) to generate pEX-pGWI-myc-Orai1.

After PCR amplification, CAD was cloned into the pCR8/GW/TOPO vector (Invitrogen) to yield pCR8-CAD. CAD and other products cloned into this vector were sequenced by using GW1 primer. Gateway LR clonase reactions (Invitrogen, Carlsbad, Calif.) were used to generate YFP-CAD and Flag-myc-CAD using the destination vectors pDS-YFP-X and pDEST-pGWI-Flag-Myc-x vector (custom-designed), respectively.

Orai1 containing an extracellular HA epitope was constructed by insertion of the sequence GSGSYPYDVPDYAGSGS between aa 207 and 208 (G-Q) in the second extracellular loop of Orai1 using an XbaI and XhoI site (Prakriya et al., 2006). This construct was used to generate Orai1-ΔN, Orai1-ΔC and Orai1-ΔN73 using PCR amplification and cloning into the pCR8/GW/TOPO vector (Invitrogen). Gateway LR clonase reactions were used to generate YFP-extHA-Orai1 constructs using the destination vector pDS-YFP-x. All constructs were confirmed by sequencing. The primers used are shown below.

For Orai1 ΔNT: Forward (SEQ ID NO:37) TCCAGCCGGACCTCGGCTCTG; Reverse (SEQ ID NO:38) CTAGGCATAGTGGCTGCCGGGCGTC. For Orai1 ΔN73: Forward (SEQ ID NO:39) GGATCCATGGCGCTGTCCTGGCGCAAG; Reverse (SEQ ID NO:40) CTAGGCATAGTGGCTGCCGGGCGTC. For Orai1 ΔCT (1-256): Forward (SEQ ID NO:41) GTCTTCGCCGTCCACTAGTACCGCTCACTGGTT; Reverse (SEQ ID NO:42) AACCAGTGAGCGGTACTAGTGGACGGCGAAGAC.

Split-Ubiquitin Yeast Two Hybrid Assay. To generate fusion proteins with the N-terminal half of ubiquitin (Nub), full-length or fragments of Orai1 were amplified by PCR and cloned into the pDL2-Nubx vector (Dualsystems Biotech). The Nub sequence in the pDL2-Nubx plasmid contains a point mutation (NubG) that abolishes the spontaneous association of Nub to the C-terminal half of ubiquitin (Cub). The CAD was subcloned into the pAMBV4 vector, which contains Cub fused to a chimeric transcription factor consisting of the DNA binding domain from LexA and the VP16 transactivation domain.

GST-CAD Construct. To generate GST fusion proteins, CAD was subcloned into pGEX 6p-1 (GE Healthcare) and the pGEX-CAD was transformed into E. coli BL21 pRIL (Stratagene). Transformants were grown in liquid cultures under ampicillin selection and isopropyl-1-thio-β-D-galactopyranoside (IPTG, 1 mM) was added at an optical density of 0.5 at 600 nm. 3 h after IPTG induction at 30° C., cells were collected by centrifugation and resuspended in PBS containing protease inhibitors. The cells were sonicated, centrifuged at 12,000 rpm for 10 min at 4° C., and the supernatant was incubated with glutathione sepharose 4B beads for 2 h. The recombinant proteins were eluted from the beads by incubation with glutathione elution buffer and subsequently dialyzed with PBS. Protein concentrations were measured by the Bradford method (Bio-Rad).

Cloning, Expression and Purification of EE-Orai1-His8 and His6-CAD. Orai1 was PCR amplified from eGFP-myc-Orai1 (described above) using primers that introduced an N-terminal EE epitope tag (sequence: EEYMPME) and a C-terminal His8 tag. The PCR product was cloned in the pVL1393 insect expression vector using BamHI and NotI restriction sites. This construct was used to produce recombinant baculovirus via recombination with the baculovirus genome (Sapphire Baculovirus; Orbigen, San Diego, Calif.) after transfection into Sf9 insect cells. Recombinant EE-Orai1-His8 was expressed by incubating Hi5 cells (Invitrogen, Carlsbad, Calif.) cultured in Insect-Xpress media (Lonza, Walkersville, Md.) with the amplified recombinant virus for 48 h at 28° C. The cells were harvested and lysed in the presence of protease inhibitors using a dounce homogenizer. Membranes were pelletted along with other cellular debris by centrifugation at 40,000×g for 1 h at 4° C., and solubilized by resuspension in a buffer containing 1% DDM (n-dodecyl-β-d-maltoside; Anatrace, Ill., USA) and gentle rotation for 1 h at 4° C. The unsolubilized material was removed by centrifugation at 40,000×g for 1 h at 4° C.

PCR-amplified CAD with an N-terminal His₆ tag was inserted in pVL1393 vector using BamHI and NotI sites, and baculovirus was generated as described above. Hi5 cells were coinfected with His₆-tagged CAD and EE-Orai1-His₈ baculoviruses and harvested after 48 h. The cells were pelletted and solubilized using 1% DDM. His₆-tagged CAD and EE-Orai1-His₈ were purified together using Ni-NTA affinity chromatography followed by anti-EE precipitation. CAD coeluted with Orai1 even after extensive washing with buffer containing 0.5 M NaCl. The complex was then passed over a Superose 6 column under the same buffer conditions described for Orai1 alone. All buffers used for CAD contained 2 mM β-mercaptoethanol (β-ME).

### Results

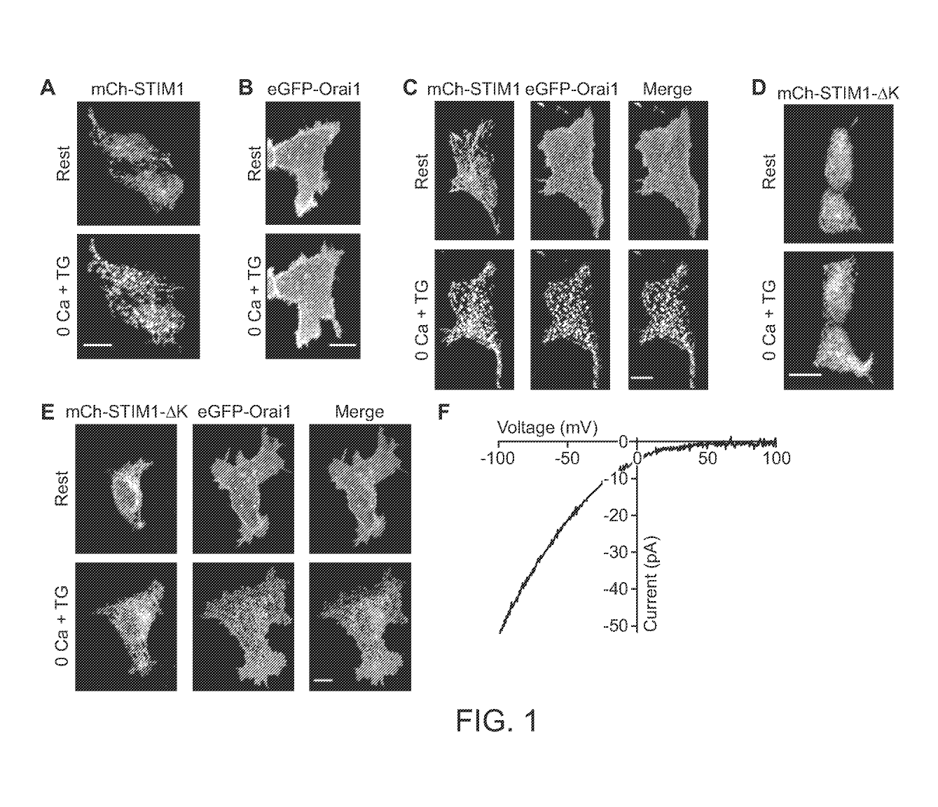

STIM1 Accumulates at ER-PM Junctions by Orai1-dependent and -independent Mechanisms. We initially investigated whether the formation of puncta by STIM1 and Orai1 following depletion of stores depends on co-expression of both proteins. When expressed by itself in HEK 293 cells, mCherry-labeled STIM1 (mCh-STIM1) formed distinct puncta after depletion of Ca²⁺ stores with thapsigargin (TG) (FIG. 1A). In contrast, GFP-myc-Orai1 expressed alone did not form puncta in response to TG (FIG. 1B), but coexpression of mCh-STIM1 restored its ability to form puncta (FIG. 1C). These results suggest that STIM1 recruitment to ER-PM junctions is independent of Orai1, whereas Orai1 recruitment to these sites depends on binding to STIM1 or a STIM1-associated protein.

To identify the regions of STIM1 that are necessary for puncta formation we investigated the role of the polybasic C-terminal domain (aa 672-685). We found that STIM1-DK failed to form puncta following store depletion when expressed alone in HEK 293 cells (FIG. 1D); however, when expressed together with Orai1, both proteins colocalized in puncta and activated I_{CRAC} after store depletion (FIG. 1E, F). These data suggest that the polybasic domain is required to localize STIM1 to ER-PM junctions in the absence of Orai1 but that a second domain recruits and activates Orai1 at these sites.

Identifying a Minimal Cytosolic Region of STIM1 that Opens the CRAC Channel. To identify the CRAC-activating domain of STIM1, we first tested a series of soluble cytosolic STIM1 fragments for their ability to activate an NFAT-dependent luciferase reporter gene (NFAT-luc). A series of constructs were generated by progressive truncation of the full-length cytosolic region of STIM1 (FIG. 2A; STIM1₂₃₄₋₆₈₅; CT-STIM1) and were transiently expressed in a HEK 293T cell line containing NFAT-luc. Because NFAT-dependent transcription requires the sustained elevation of intracellular Ca²⁺ ([Ca²⁺]ᵢ) combined with a phorbol ester to activate protein kinase C (PKC), treatment of cells bearing only NFAT-luc with phorbol 12-myristate 13-acetate (PMA; 1 μM) does not stimulate luciferase production. However, PMA in conjunction with 1 μM TG, which activates Ca²⁺ entry through endogenous CRAC channels, activates NFAT-luc significantly (FIG. 2B). Therefore we compared luciferase production in the presence of PMA with that in PMA+TG to assess the ability of STIM1 fragments to activate endogenous CRAC channels.

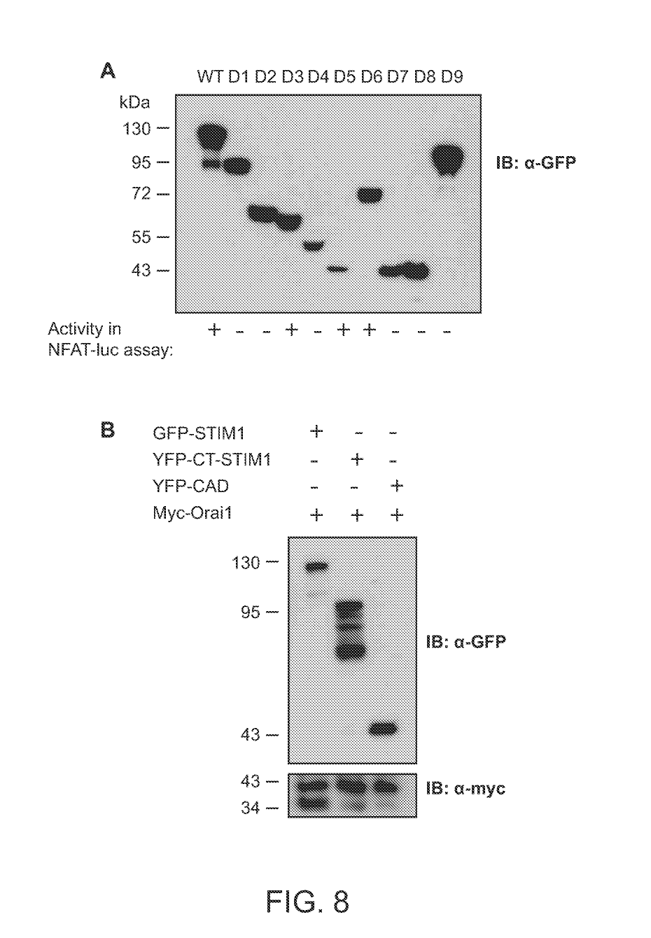

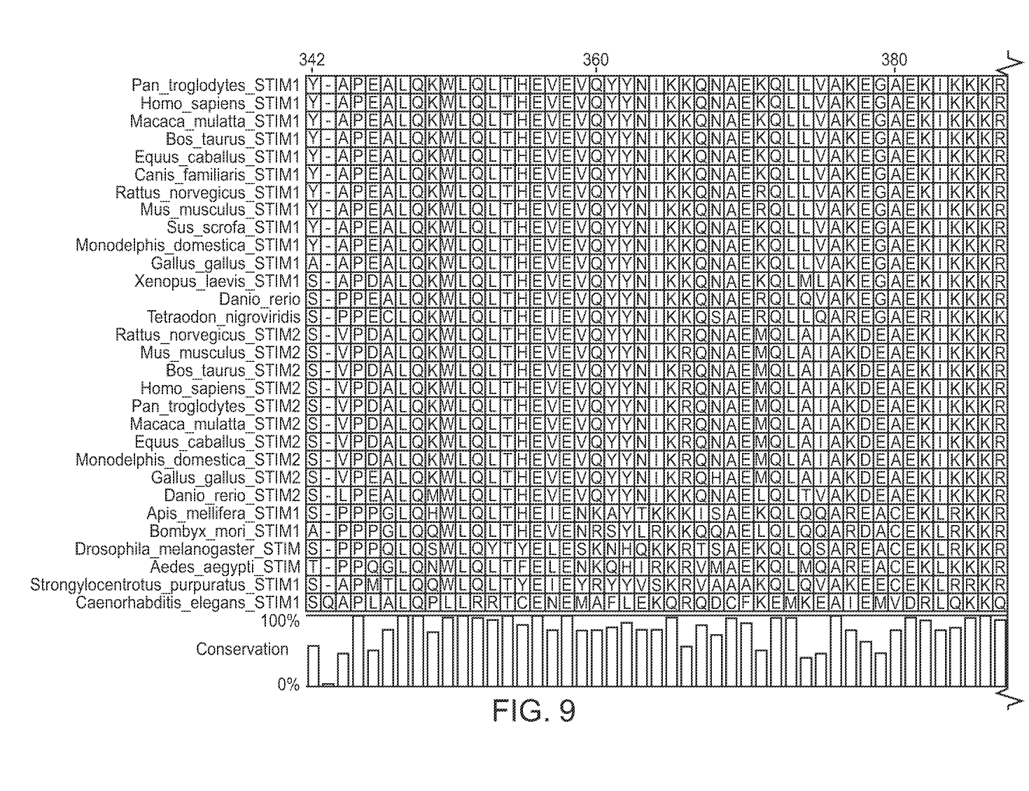

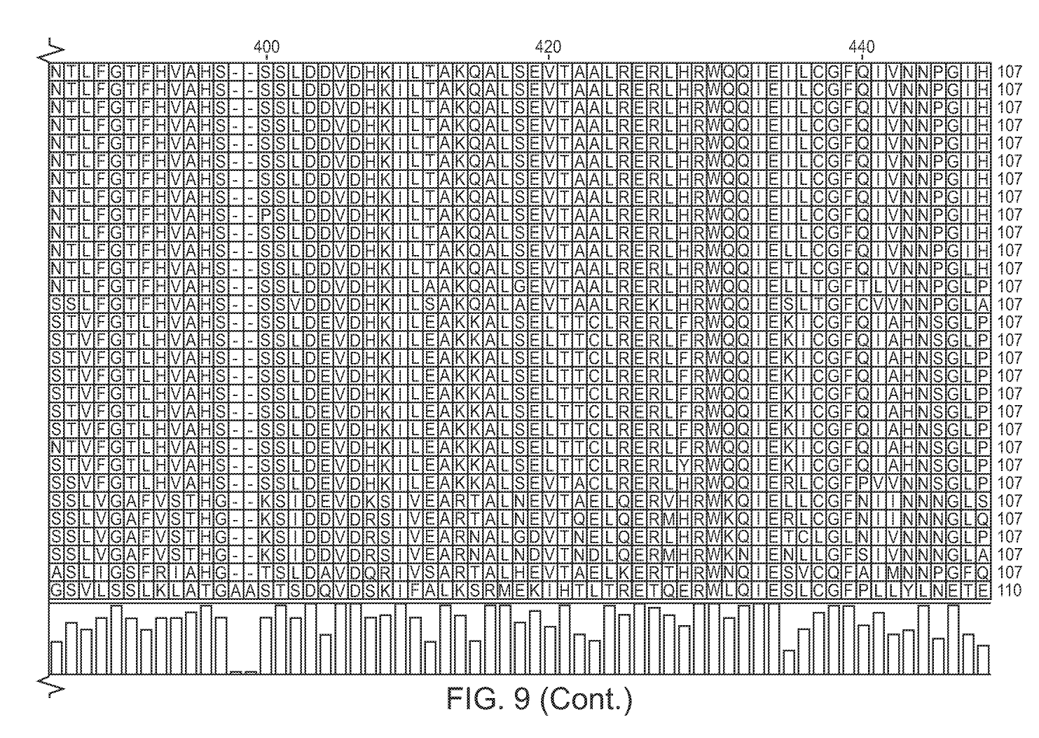

While CT-STIM1 did not activate the NFAT reporter gene with PMA alone, truncations of either the C- or N-terminus of this protein generated several active STIM1 peptides (D3, D5, D6). By making additional truncations we identified STIM1₃₄₂₋₄₄₈ (D5) as the minimal peptide that was sufficient to activate NFAT-luc; we will refer to this domain hereafter as the CRAC activation domain (CAD). Western analysis showed that the inactivity of peptides D1, D2, D4, and D7-9 was not due to inadequate expression (FIG. 8A). The CAD encompasses a putative coiled-coil and part of the ERM domain of STIM1, is highly conserved among vertebrates and invertebrates from C. elegans to H. sapiens and is virtually identical to a sequence in STIM2, another ER Ca²⁺ sensor that controls CRAC channel activation (FIG. 9).

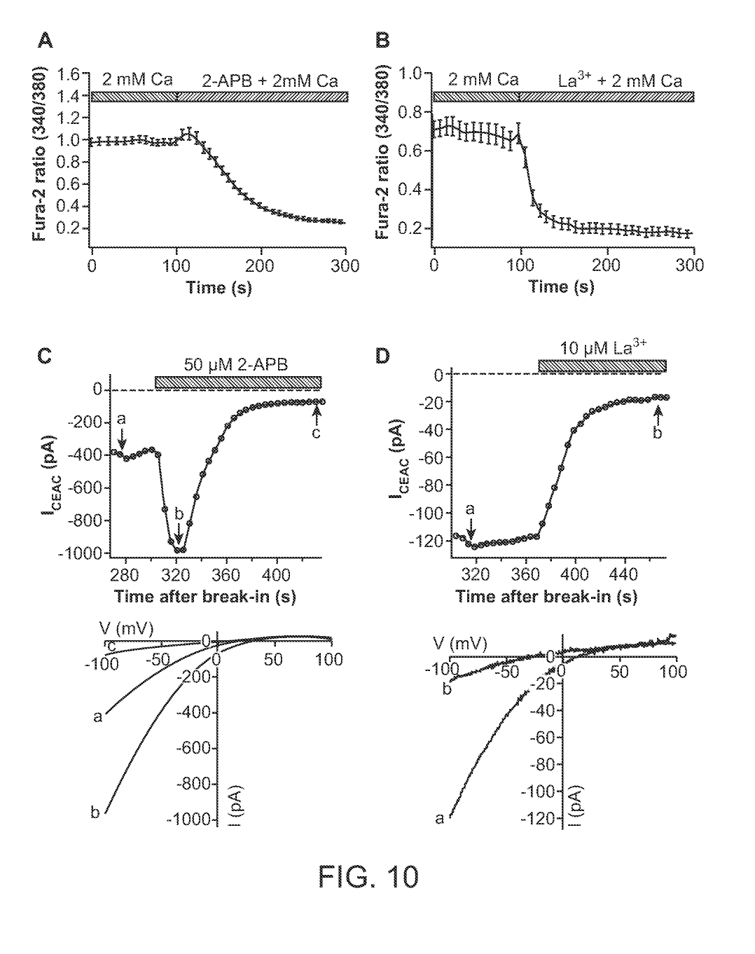

To determine if CAD activates store operated Ca²⁺ influx in cells, we measured [Ca²⁺]ᵢ in HEK 293 cells expressing CAD and Orai1. CAD evoked a sustained [Ca²⁺]ᵢ elevation that was dependent on extracellular Ca²⁺ (FIG. 2C), was suppressed by CRAC channel inhibitors like 2-APB and 10 μM La³⁺ (FIG. 10 A, B), and was not observed in cells coexpressing a dominant-negative nonconductive Orai1 mutant (Orai1_{E106A}; FIG. 2C). Importantly, CAD activated Ca²⁺ entry without depleting intracellular stores, because the Ca²⁺ released by TG in Ca²⁺-free media was similar in CAD-expressing and untransfected cells (FIG. 2C). These results demonstrate that CAD elevates [Ca²⁺]ᵢ by activating CRAC channels independently of store depletion.

As a definitive test for CRAC channel activation, we conducted whole-cell patch clamp recordings from HEK 293 cells transiently transfected with myc-Orai1 and either YFP-STIM1 or YFP-CAD. In cells expressing full length YFP-STIM1, I_{CRAC} appeared over several minutes following break-in, consistent with the typical slow activation seen in response to passive store depletion (FIG. 2D). I_{CRAC} showed the characteristic inwardly-rectifying current-voltage relation (FIG. 2D, right), inhibition by La³⁺ and modulation by 2-APB. In contrast, inward current was present from the moment of break-in in cells expressing YFP-CAD (FIG. 2E). This current was identified as I_{CRAC} based on its dependence on extracellular Ca²⁺, inward rectification, sequential enhancement and inhibition by 2-APB, and inhibition by La³⁺ (FIG. 2E and FIG. 10 C, D). I_{CRAC} was not observed at break-in in HEK 293 cells transfected with YFP or YFP-CAD in the absence of Orai1 (FIG. 2F), consistent with the low level of endogenous STIM1 and Orai1 in these cells. Interestingly, the current activated by CAD differed from native I_{CRAC} in that it lacked fast Ca²⁺-dependent inactivation. Inactivation was restored by addition of STIM1 residues carboxy-terminal to CAD.

Transfection of HEK 293 cells with YFP-CT-STIM1+myc-Orai1 failed to activate constitutive I_{CRAC} (FIG. 2F) or elevate resting [Ca²⁺]ᵢ, consistent with the lack of activity in the NFAT-luciferase assay. CT-STIM1 was inactive even though it was expressed at comparable levels to CAD or wild-type STIM1 (WT-STIM1; FIG. 8 B). While coexpression of CT-STIM1+Orai1 did not elevate resting [Ca²⁺]ᵢ in HEK 293 cells, it did so in HEK 293T cells, which express large T antigen and express proteins at substantially higher levels than HEK 293 cells. Our studies demonstrate that CAD is a much more potent activator of Orai1 than CT-STIM1, based on its ability to activate I_{CRAC} comparably to WT-STIM1 at expression levels at which CT-STIM1 activity is undetectable (FIG. 2F).

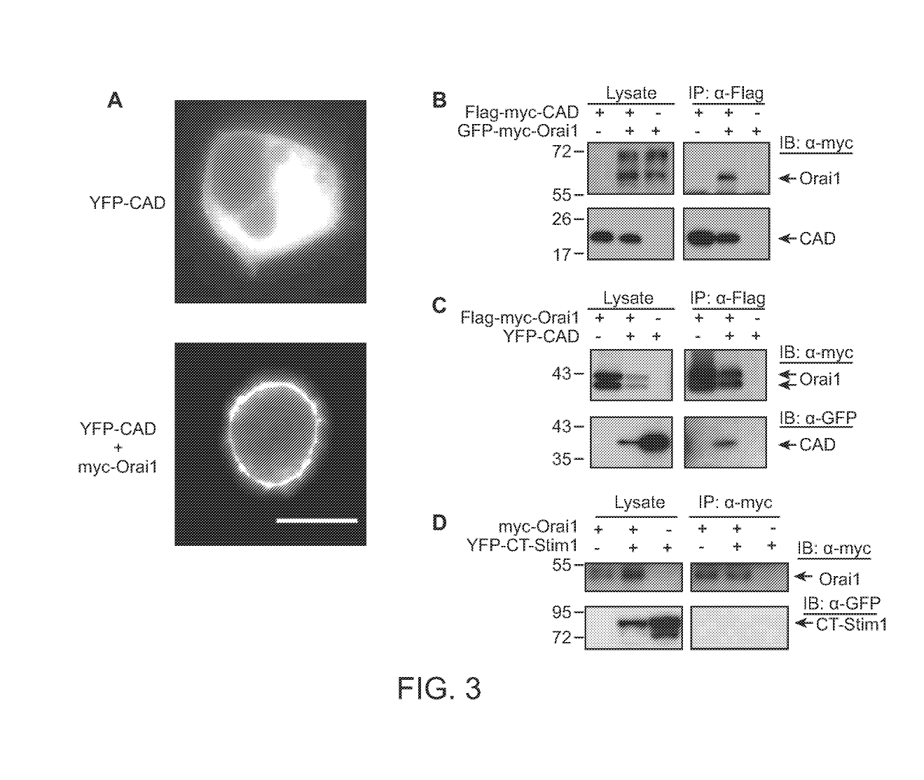

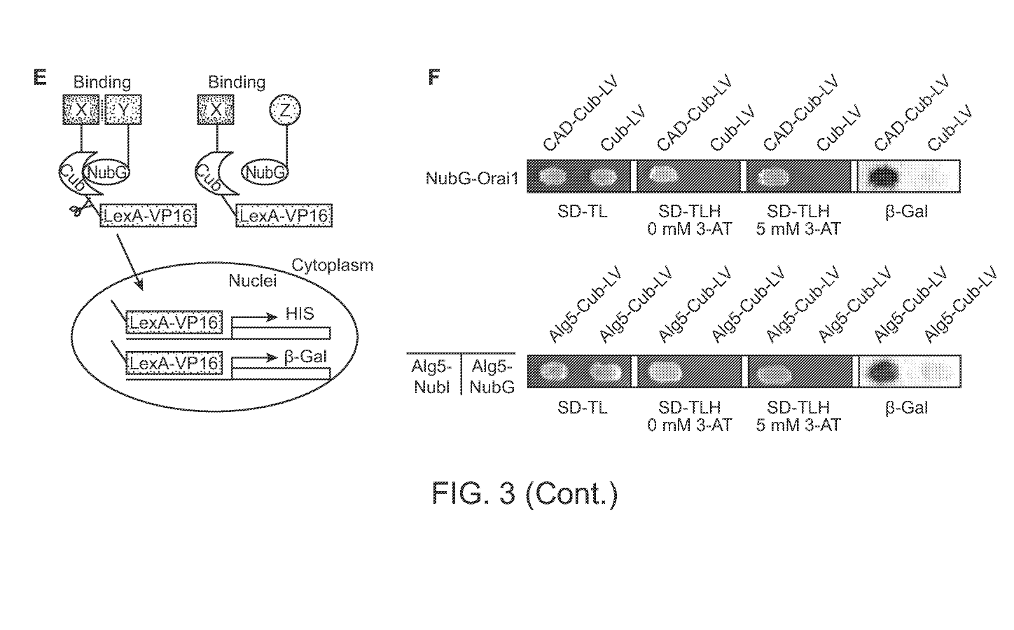

CAD Associates with Orai1 in vivo and in vitro. Because cytosolic CAD is a potent activator of Orai1 channels and is not associated with the ER, we hypothesized that CAD binds to Orai1. To test this idea, we first expressed YFP-CAD with or without myc-Orai1 in HEK 293 cells and examined its intracellular localization. In the absence of Orai1, YFP-CAD was localized diffusely throughout the cytoplasm, but the introduction of Orai1 led to a dramatic recruitment of YFP-CAD to the plasma membrane, suggesting that the two proteins form a complex (FIG. 3A). To provide additional evidence that CAD and Orai1 are part of the same protein complex we expressed Flag-tagged CAD and GFP-tagged Orai1 in HEK 293T cells and immunoprecipitated CAD with anti-Flag antibodies. GFP-myc-Orai1 was detected in the immunoprecipitates from cells co-expressing CAD and Orai1 but not from cells expressing CAD or Orai1 alone (FIG. 3B).

The reciprocal experiment, in which Flag-tagged Orai1 was immunoprecipitated using anti-Flag antibodies and YFP-CAD was detected by Western blotting, confirmed that CAD co-immunoprecipitates with Orai1 only when the two proteins are coexpressed (FIG. 3C). Thus, CAD and Orai1 form a protein complex in mammalian cells. Under the same conditions (150 mM salt, 1% Triton X-100) we were unable to detect any interaction between CT-STIM1 and Orai1 (FIG. 3D), suggesting that the affinity of the full-length cytoplasmic domain of STIM1 for Orai1 is much weaker than that of the isolated CAD. This result may explain why CT-STIM1 is a weak activator of CRAC channels and suggests that CAD is not exposed in CT-STIM1.

As a third test for association of CAD and Orai1 in vivo, and to map the interaction domains of Orai1 we introduced both proteins into a yeast split ubiquitin interaction system. In this system, interaction of the two test proteins reunites the N- and C-terminal fragments of ubiquitin, releasing a LexA-VP16 transcriptional activator that enters the nucleus and activates reporter genes (FIG. 3E). Fusion proteins of Orai1 and the N-terminal fragment of ubiquitin (NubG-Orai1), and of CAD and the C-terminal fragment of ubiquitin fused to LexA-VP16 (CAD-Cub-LV) were introduced into a yeast strain containing LexA His and b-galactosidase reporter genes. Yeast containing both the NubG-Orai1 and CAD-Cub-LV survived on His selection plates and produced significant levels of b-gal, whereas yeast expressing NubG-Orai1 and the Cub-LV domain alone did not (FIG. 3F), indicating that CAD and Orai1 interact with each other in a heterologous system.

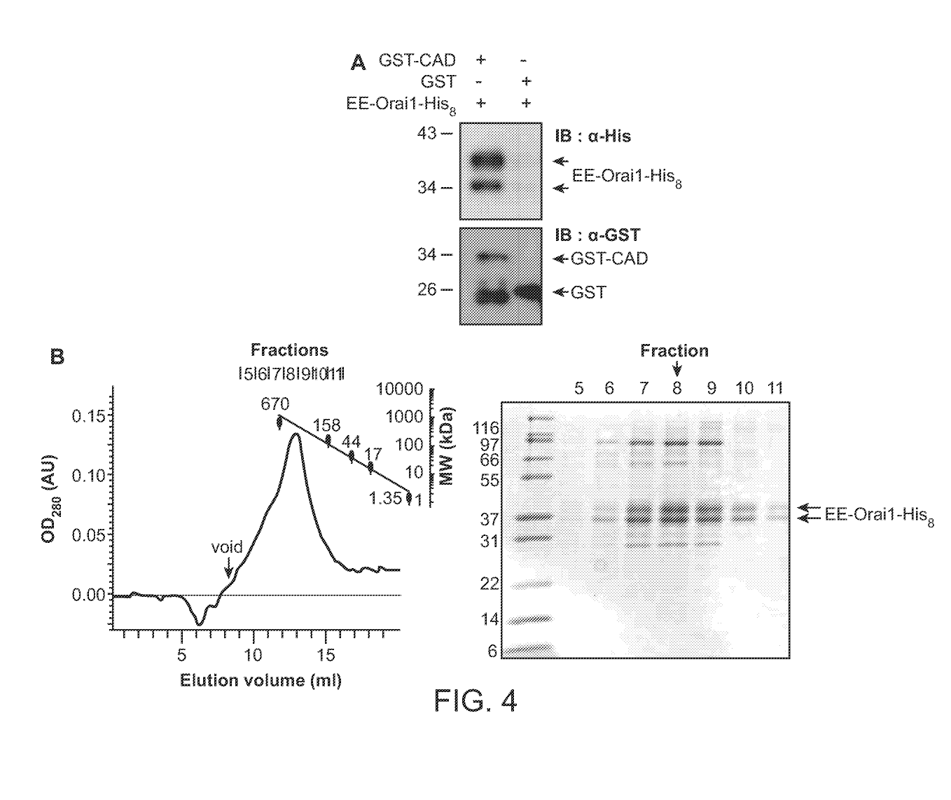

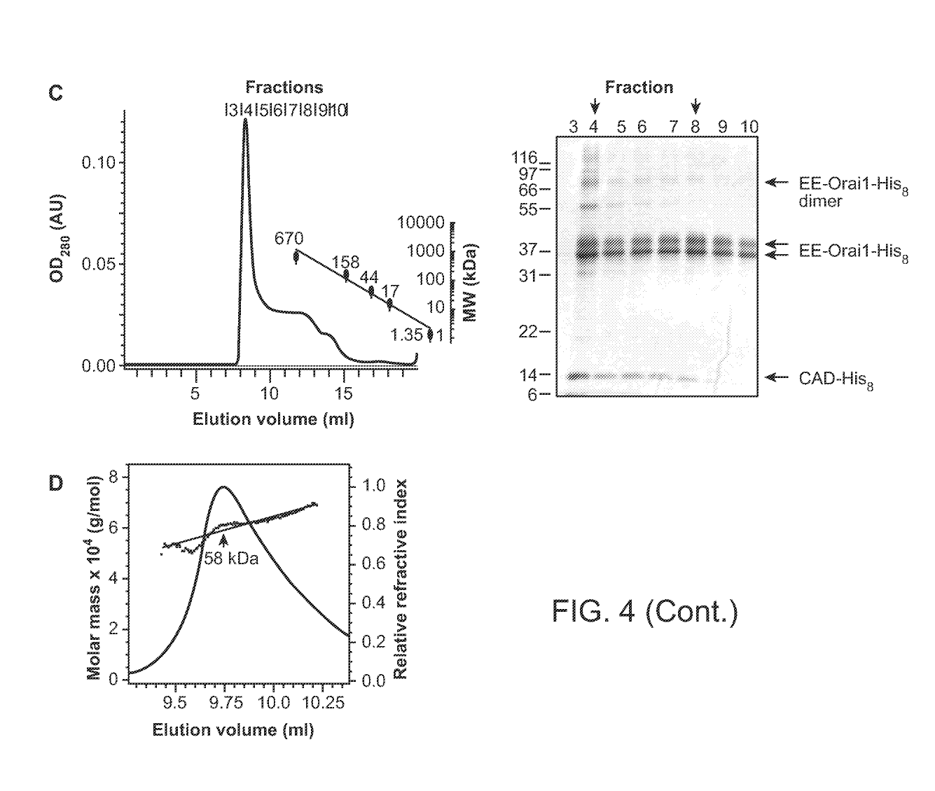

CAD Binds Directly to Orai1. To test for direct binding between the CAD and Orai1 we generated a GST-tagged CAD peptide in E. coli and an Orai1 protein containing C-terminal octa-histidine and N-terminal EEYMPME (“EE”) tags in insect Hi5 cells. We incubated purified GST-CAD or GST alone with purified EE-Orai1-His₈ and used glutathione beads to precipitate the complexes. GST-CAD co-precipitated Orai1, while GST alone did not, indicating that CAD binds directly to Orai1 in vitro (FIG. 4A).

To examine the size of the protein complexes generated by the interaction of Orai1 and CAD, we prepared extracts from Hi5 cells expressing EE-Orai1-His₈ alone or in combination with CAD-His₆ and analyzed them by size exclusion chromatography. Orai1 and CAD proteins were first affinity-purified with Ni²⁺-NTA and Orai1 was then immunoprecipitated with anti-EE antibodies. Size exclusion chromatography of Orai1 alone revealed a monodisperse peak with an apparent mass of ˜290 kD (FIG. 4B, left) consistent with Orai1 forming multimers in the absence of CAD. Analysis of the eluate fractions by SDS-PAGE revealed a doublet of ˜37 kD (FIG. 4B, right) representing glycosylated and unglycosylated forms of EE-Orai1-His₈ as determined by the ability of tunicamycin to collapse the top band to the lower band (data not shown). In contrast to Orai1 alone, EE-Orai1-His₈ purified from cells coexpressing CAD-His₆ eluted largely in the void volume, consistent with the formation of a large protein complex of molecular weight>10 MDa (FIG. 4C).

The complex was stable in 0.5 M NaCl, suggesting a high-affinity hydrophobic interaction of multiple CAD and Orai1 proteins. Importantly, complex formation did not involve significant amounts of additional proteins, indicating that the interaction between CAD and Orai1 is direct (FIG. 4C). The excess free CAD-His₆, isolated by gel filtration after Ni²⁺-NTA purification of the complex, was analyzed by multi-angle light scattering (MALS) which indicated a molecular weight of ˜58 kD, or 4.4 times the predicted weight of 13.2 kD for the CAD-His₆ monomer (FIG. 4D). Together, these results show that Orai1 exists as a multimer in the absence of CAD, and CAD forms a tetramer in free solution that links together multiple Orai1 multimers.

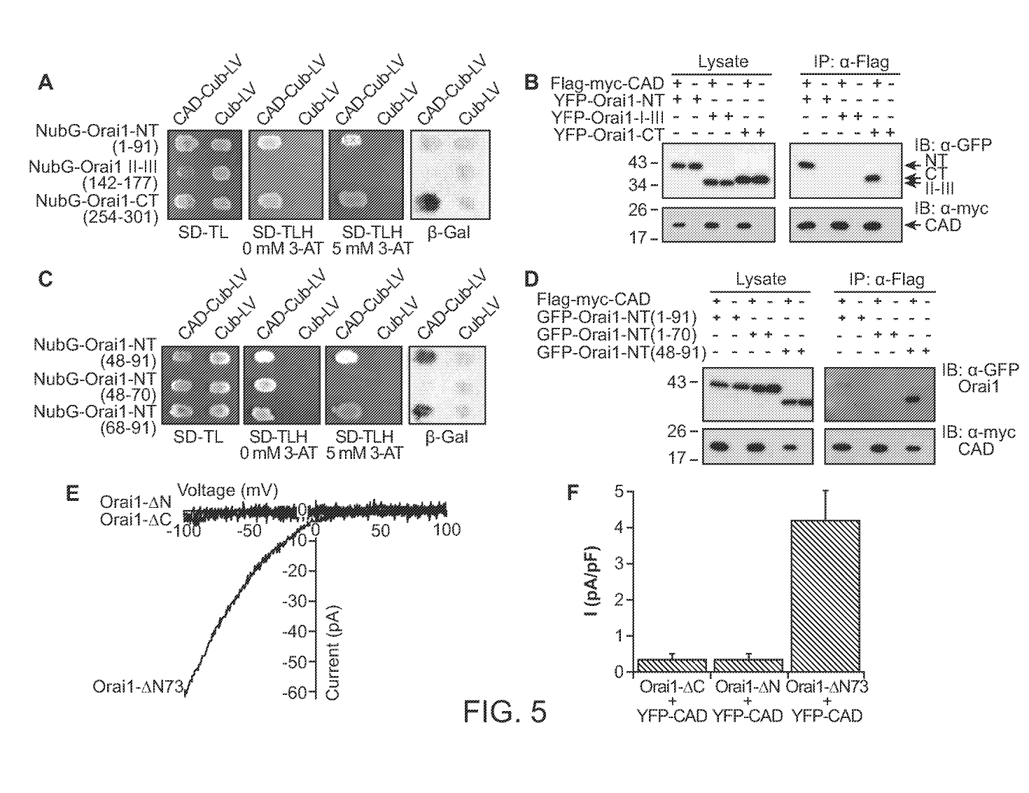

STIM1-CAD BINDS TO THE N- AND C-TERMINI OF ORAI1. To identify regions of Orai1 important for activation by CAD, we characterized the binding of CAD to Orai1 using two approaches. First we expressed fusions of NubG with the N-terminus, the II-III cytoplasmic loop and the C-terminus of Orai1 in yeast together with different CAD-Cub-LV constructs. Survival of colonies in selection media and production of b-gal revealed that CAD binds to the N- and C-terminus of Orai1 but not to the II-III loop or to the C ubiquitin fragment alone (FIG. 5A). Based on the level of b-gal activity, CAD appears to interact with the Orai1 C-terminus with a higher affinity than with the N-terminus. We next expressed fusions of YFP with the N-terminus, the II-III loop and the C-terminus of Orai1 in HEK 293T cells together with Flag-myc-CAD. Immunoprecipitation of Flag-myc-CAD followed by Western blotting revealed that CAD interacts with the N- and C-terminus of Orai1 but not with the II-III loop (FIG. 5B).

To explore the interaction between the N-terminus of Orai1 and CAD in more detail we mapped the subregion within the N-terminus of Orai1 that is responsible for CAD binding, using both the split ubiquitin and HEK 293T co-immunoprecipitation assays. In the yeast assay, CAD interacted strongly with Orai1₄₈₋₉₁ and Orai1₆₈₋₉₁ but not with Orai1₄₈₋₇₀. Similarly, in the HEK cell assay CAD co-immunoprecipitated with Orai1₄₈₋₉₁ but not with Orai1₁₋₇₀, suggesting that CAD binds to the region of aa 70-91 (FIG. 5D). The interaction between CAD and Orai1₄₈₋₉₁ was significantly stronger than that between CAD and the full-length N-terminus of Orai1 (FIG. 5D, right), suggesting that aa 1-48 reduce the affinity between CAD and the isolated Orai1 N-terminus.

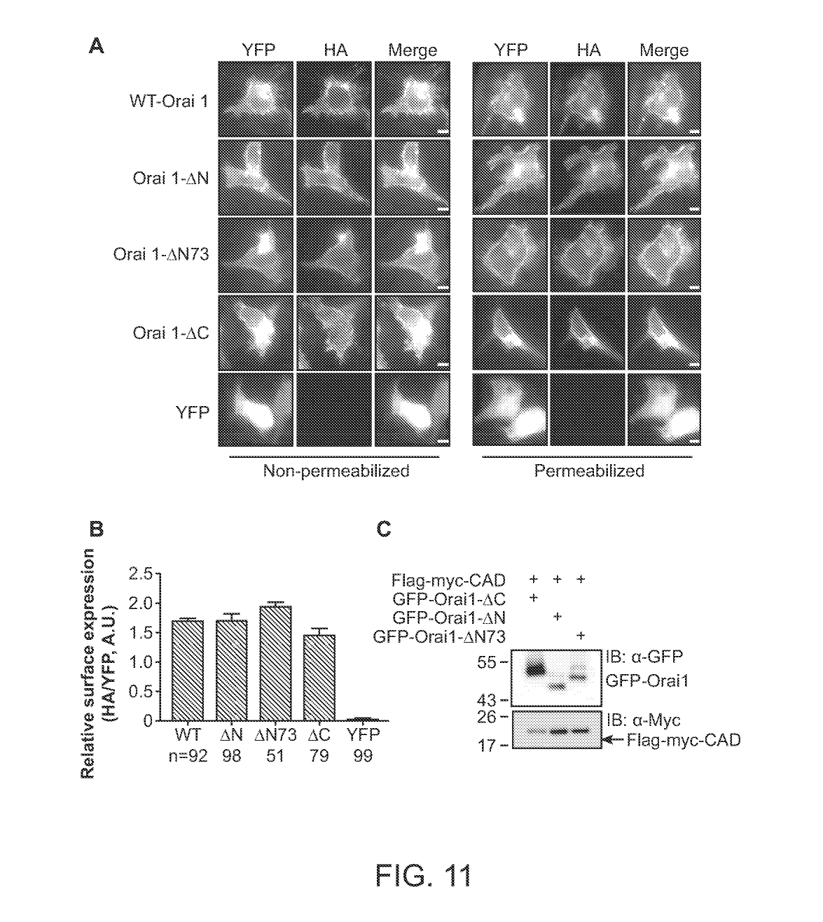

We next tested the function of the CAD-binding regions of Orai1 using whole-cell recording. We introduced CAD into HEK 293 cells together with Orai1 lacking the full N-(Orai1-ΔN) or C-terminus (Orai1-ΔC) or the initial 73 residues of the N-terminus (Orai1-ΔN73) preceding the CAD binding site. Western blotting and immunostaining of Orai1 containing an extracellular HA epitope confirmed that these mutations do not alter the expression or cell surface localization of the channels (FIG. 11). CAD constitutively activated I_{CRAC} in cells expressing Orai1-ΔN73 but not in cells expressing Orai1-ΔN or Orai1-ΔC (FIG. 5E, F), showing that the N- and C-termini of Orai1 are both necessary for activation by CAD but that aa 1-73 are not absolutely required.

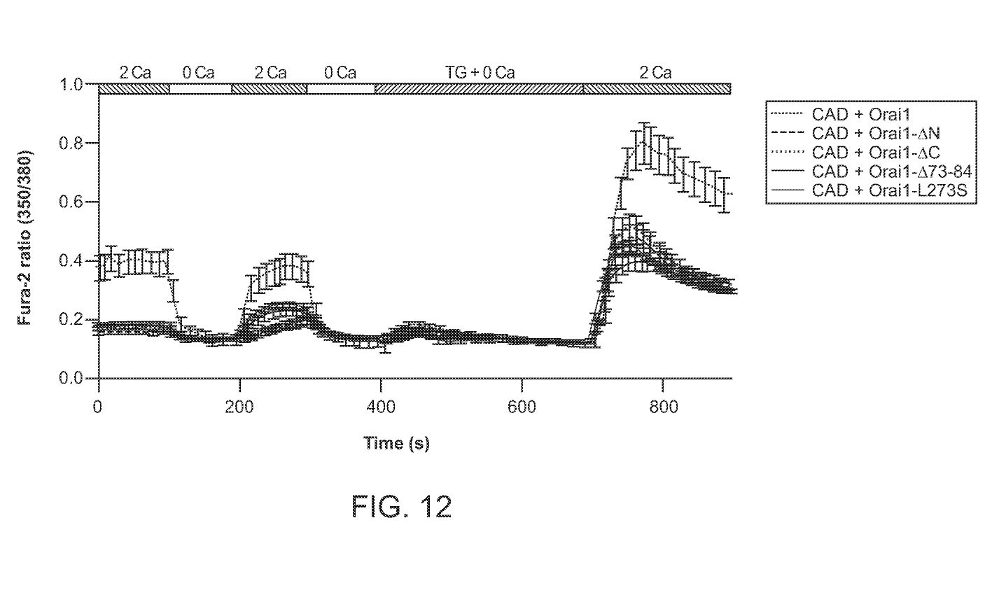

Furthermore, deletion of aa 73-84 from Orai1 suppressed CAD-induced Ca²⁺ influx (FIG. 12). Taken together with the results of FIGS. 5C and D, these findings suggest that CAD binding to the C-terminus of Orai1 and the membrane-proximal region of the Orai1 N-terminus is required to activate the CRAC channel.

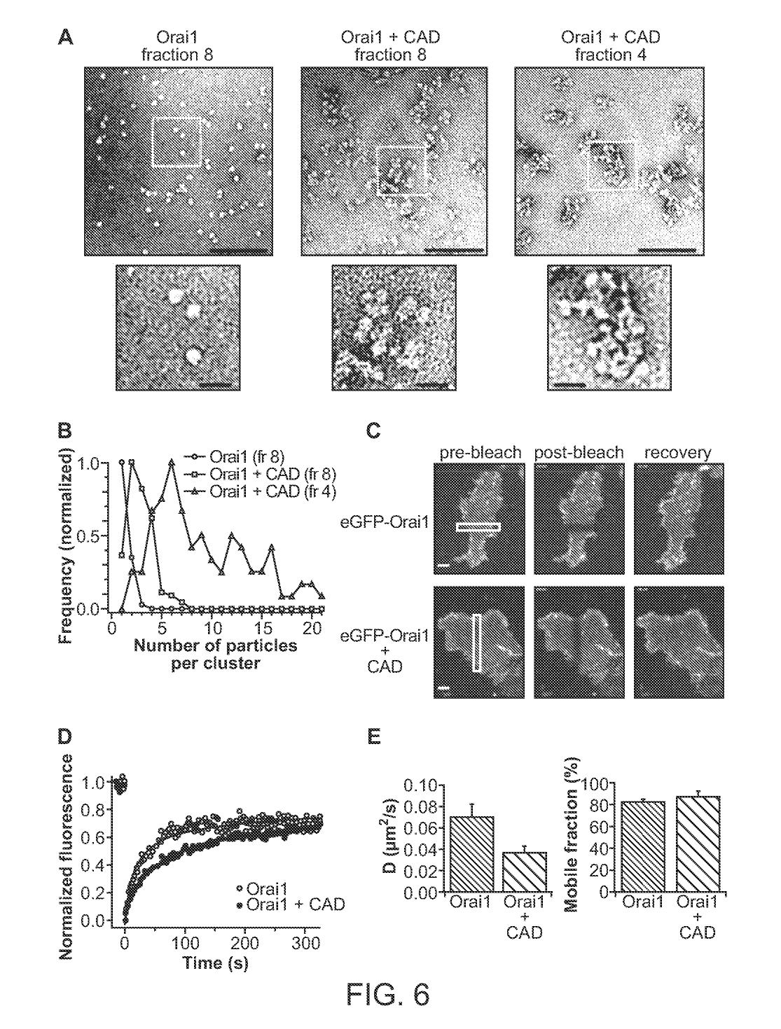

CAD Clusters CRAC Channels. The large size of the CAD/Orai1 complex indicates that CAD clusters Orai1. To investigate the nature of these complexes and test for non-specific aggregation, we examined purified material from the gel filtration column by negative stain single-particle electron microscopy (FIG. 6A, B). Analysis of purified Orai1 alone revealed primarily particles of 8-10 nm diameter, presumably representing single CRAC channels, with a low frequency of pairs and triplets. In contrast, in the presence of CAD we observed clusters of Orai1 unitary particles that increased in frequency and size with increasing molecular weight of the column eluates. Taken together with the MALS results these images suggest that tetramers of CAD bind to multiple sites on CRAC channels to create these clusters.

To test whether CAD also clusters CRAC channels in intact cells, we conducted fluorescence recovery after photobleaching (FRAP) experiments of GFP-Orai1 expressed alone or with CAD in HEK 293 cells. Our FRAP measurements of GFP-Orai1 alone suggest that 83±2% of the channels are mobile in the membrane with an effective diffusion coefficient (D) of 0.070±0.011 μm²/s (n=9, FIG. 6C-E). In cells co-expressing GFP-Orai1 and CAD, the mobile fraction was unchanged (87±4%) but Orai1 diffusion was slowed by a factor of two (D=0.036±0.006 μm²/s, n=6). Thus, CAD does not appear to anchor CRAC channels to an immobile substrate, but does slow their diffusion significantly, consistent with CAD-induced clustering of Orai1 in the cell membrane.

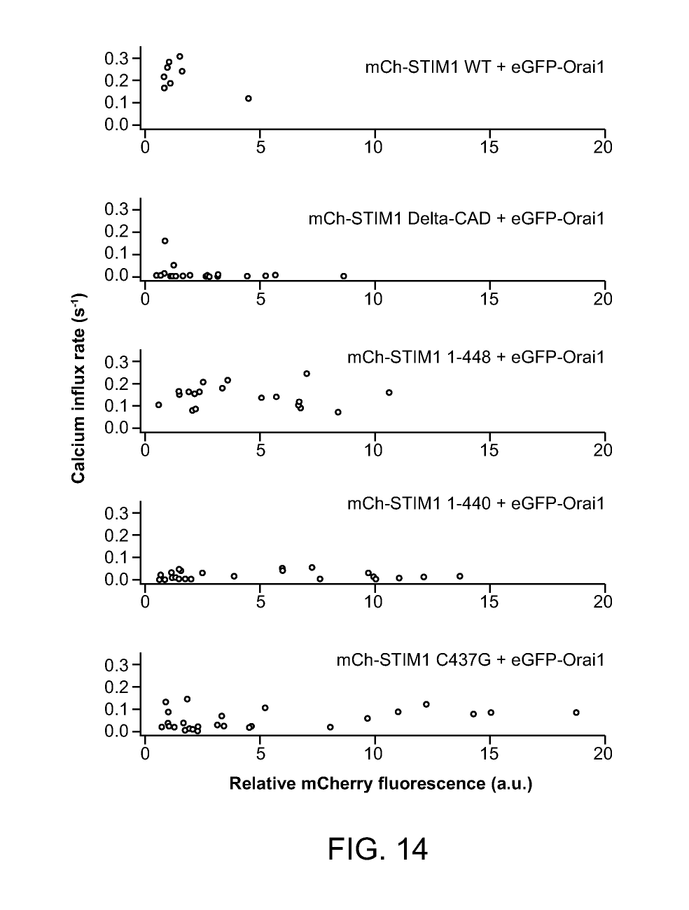

The Role of CAD in Orai1 Binding and Activation by STIM1. A key question is whether CAD is responsible for the clustering and activation of CRAC channels by full-length STIM1 following store depletion. To address this, we measured the ability of mCherry-tagged STIM1 variants containing CAD mutations to co-cluster with eGFP-Orai1 and activate Ca²⁺ entry in response to TG (FIG. 7). STIM1-Orai1 colocalization was quantified as the fraction of total STIM1 and Orai1 fluorescence that was recruited to regions of high fluorescence covariance (FIG. 13), while Ca²⁺ influx was measured with single cell Ca²⁺ imaging. For each set of experiments we measured mCherry and GFP fluorescence to confirm that differences in the activity of the various STIM1 constructs were not due to differences in expression level (FIG. 14).

We first deleted the CAD from WT-STIM1 (STIM1-DCAD) and found that when expressed with eGFP-Orai1, mCh-STIM1-DCAD failed to form puncta, cluster Orai1 or activate Ca²⁺ entry in response to TG (FIG. 7A, E, F). Moreover, in the NFAT-luciferase assay, STIM1-DCAD inhibited NFAT activation by endogenous channels following treatment with TG+PMA (FIG. 2B), suggesting that it forms non-functional oligomers with endogenous STIM1. These results show that the CAD is required for STIM1 to form puncta and to recruit and activate Orai1 at ER-PM junctions.

To test whether the CAD in STIM1 can function independently of the residues C-terminal to it, we truncated STIM1 from the C-terminus to the end of the CAD (STIM1₁₋₄₄₈). When coexpressed with Orai1, STIM1₁₋₄₄₈ behaved much like WT-STIM1; it was distributed throughout the ER of resting cells, and following store depletion it formed puncta with Orai1 and activated Ca²⁺ entry (FIG. 7B, E), though both puncta and Ca²⁺ entry were somewhat less pronounced than with WT-STIM1 (FIG. 7F). Therefore the region of STIM1 carboxy-terminal to CAD is not absolutely required for CRAC channel binding or activation, although it may make a minor contribution to both.

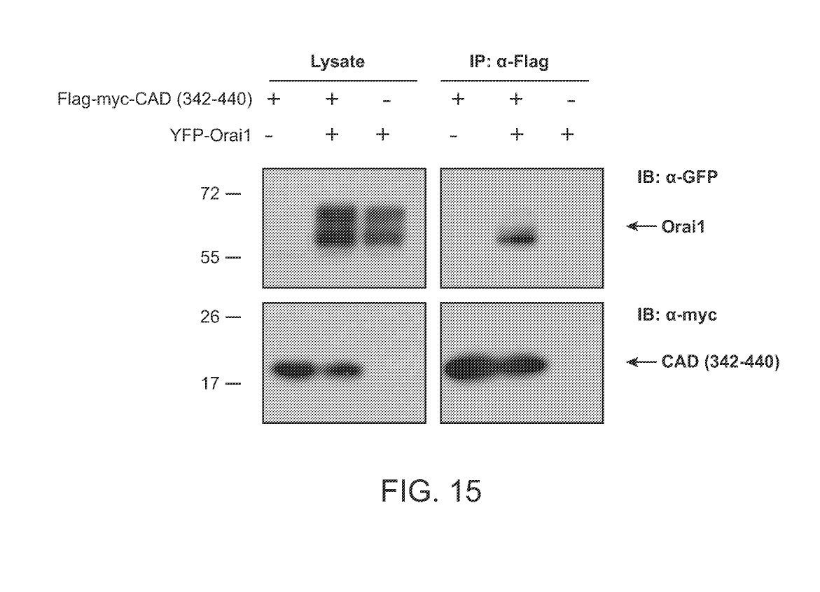

Deleting the last 8 aa from the CAD (342-440) peptide eliminates its ability to activate CRAC channels (FIG. 2B, C). We therefore tested whether this mutation also prevents STIM1₁₋₄₄₈ from activating Orai1 by deleting the final 8 aa to generate STIM1₁₋₄₄₀. STIM1₁₋₄₄₀ failed to activate SOCE after store depletion (FIG. 7E, F), supporting the idea that CAD function is required for CRAC channel activation by STIM1. Surprisingly, however, STIM1₁₋₄₄₀ retained the ability to form puncta and cluster Orai1, though to a lesser extent than WT-STIM1 (FIG. 7C, F). Consistent with this result, co-immunoprecipitation experiments revealed that the CAD 342-440 retained the ability to bind to Orai1 (FIG. 15) even though it cannot activate it. We obtained a similar result with a STIM1 C437G mutant, which formed pronounced puncta with Orai1 but only marginally activated SOCE (FIG. 7D-F). Together, these data indicate that clustering of CRAC channels by itself is not sufficient to cause channel opening.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

1. A method of modulating activity of a calcium release-activated calcium (CRAC) channel in a cell, the method comprising:
altering the activity of said CRAC channel by contacting with a modulatory polypeptide.

2. The method of claim 1, wherein the modulatory polypeptide is an enhancing polypeptide consisting or consisting essentially of SEQ ID NO:1 or fragments and derivatives thereof.

3. The method of claim 1, wherein the modulatory polypeptide is an inhibitory polypeptide consisting or consisting essentially of SEQ ID NO:2, SEQ ID NO:3, SEQ ID NO:4, SEQ ID NO:5, or SEQ ID NO:6 or fragments and derivatives thereof.

4. The method of claim 1, wherein the polypeptide is fused to a permeant domain.

5. The method of claim 1, wherein the polypeptide is encoded by an expression vector introduced into said cell.

6. The method of claim 1, wherein the cell is a T cell.

7. The method of claim 1, wherein the cell is a human cell.

8. The method of claim 1, wherein said altering step is performed in vitro.

9. The method of claim 1, wherein said altering step is performed in vivo.

10. The method according to claim 6, wherein said cell is involved in a T cell mediated immune dysfunction.

11. A method for screening for a candidate agent capable of modulating the interaction between STIM and Orai proteins, using CAD or CAD-like proteins and Orai or Orai-derived polypeptides that may contain detectable domains, said method comprising:
providing a cell, wherein CAD or CAD-like protein is transiently or stably expressed with an Orai or Orai-derived polypeptide;
contacting said cell with a candidate agent;
assessing changes in the localization of the CAD or CAD-like protein in said cell as a result of said contacting with a candidate agent;
identifying said candidate agent as a modulator of the interaction between STIM and Orai proteins in said cell as a result of said assessing.

12. The method of claim 11, wherein said candidate agent is selected from one or more of: small molecule, protein, polypeptide, polysaccharide or polynucleotide, and wherein said candidate agent is identified as an inhibiting modulator of the interaction between STIM and Orai proteins when the assessed change in the localization of the CAD or CAD-like protein results in a reduced amount of CAD or CAD-like protein at the cell membrane.

13. The method of claim 12, wherein said candidate agent is selected from one or more of: small molecule, protein, polypeptide, polysaccharide or polynucleotide, and wherein said candidate agent is identified as an enhancing modulator of the interaction between STIM and Orai proteins when the assessed change in the localization of the CAD or CAD-like protein results in an increased amount of CAD or CAD-like protein at the cell membrane.

14. A method for screening for a candidate agent capable of modulating the interaction between STIM and Orai proteins, using CAD or CAD-like proteins and Orai or Orai-derived polypeptides that may contain detectable domains, said method comprising:
providing CAD, CAD-like, Orai and Orai-derived polypeptides that were produced recombinantly or synthetically;
contacting a CAD or CAD-like polypeptide with an Orai or Orai-derived polypeptide and the candidate agent or contacting an Orai or Orai-derived polypeptide with a CAD or CAD-like polypeptide and the candidate agent;
determining the binding between said CAD or CAD-like proteins and said Orai or Orai-derived polypeptides in the presence of the candidate agent;
identifying said candidate agent as a modulator of the interaction between STIM and Orai proteins in said cell as a result of said binding.

15. The method of claim 14, wherein said candidate agent is selected from one or more of: small molecule, protein, polypeptide, polysaccharide or polynucleotide, and wherein said candidate agent is identified as an inhibiting modulator of the interaction between STIM and Orai proteins when the candidate agent decreases the binding between CAD or CAD-like protein and Orai or Orai-derived polypeptide.

16. The method of claim 14, wherein said candidate agent is selected from one or more of: small molecule, protein, polypeptide, polysaccharide or polynucleotide, and wherein said candidate agent is identified as an enhancing modulator of the interaction between STIM and Orai proteins when the candidate agent increases the binding between CAD or CAD-like protein and Orai or Orai-derived polypeptide.

17. The method of claim 14, wherein a compound library is screened for a candidate agent capable of binding to CAD, CAD-like, Orai or Orai-derived polypeptides and, thus, modulating the interaction between STIM and Orai proteins.

18. A method for screening for a candidate agent capable of modulating the interaction between STIM and Orai proteins, using CAD or CAD-like proteins, said method comprising:
providing CAD and CAD-like polypeptides that were produced recombinantly or synthetically and that may contain detectable domains;
providing CAD and CAD-like polypeptides that were produced recombinantly or synthetically and that may not contain detectable domains;
contacting said CAD or CAD-like polypeptides that may contain detectable domains with said CAD or CAD-like polypeptides that may not contain detectable domains and the candidate agent;
determining the binding between said CAD or CAD-like proteins that may contain detectable domains and said CAD or CAD-like proteins that may not contain detectable domains in the presence of the candidate agent;
identifying said candidate agent as a modulator of the interaction between STIM and Orai proteins as a result of said binding.

19. The method of claim 18, wherein said candidate agent is selected from one or more of: small molecule, protein, polypeptide, polysaccharide or polynucleotide, and wherein said candidate agent is identified as an inhibiting modulator of the interaction between STIM and Orai proteins when the candidate agent decreases the binding between CAD or CAD-like protein that may contain detectable domains and said CAD or CAD-like proteins that may not contain detectable domains.

20. The method of claim 18, wherein said candidate agent is selected from one or more of: small molecule, protein, polypeptide, polysaccharide or polynucleotide, and wherein said candidate agent is identified as an enhancing modulator of the interaction between STIM and Orai proteins when the candidate agent increases the binding between CAD or CAD-like protein that may contain detectable domains and said CAD or CAD-like proteins that may not contain detectable domains.

21. A method for screening for a candidate agent capable of modulating the interaction between STIM and Orai proteins, using CAD or CAD-like proteins and Orai or Orai-derived polypeptides that may contain detectable domains which are able to function as nonradioactive energy acceptors or energy donors, said method comprising:
providing a cell, wherein CAD or CAD-like protein is transiently or stably expressed with an Orai or Orai-derived polypeptide;
contacting said cell with a candidate agent;
assessing changes in energy levels of the CAD or CAD-like protein and the Orai or Orai-derived polypeptide due to energy transfer;
identifying said candidate agent as a modulator of the interaction between STIM and Orai proteins as a result of said assessing.

22. The method of claim 21, wherein said candidate agent is selected from one or more of: small molecule, protein, polypeptide, polysaccharide or polynucleotide, and wherein said candidate agent is identified as an enhancing modulator of the interaction between STIM and Orai proteins when the candidate agent increases the energy transfer between CAD or CAD-like proteins and Orai or Orai-derived polypeptides.

23. The method of claim 21, wherein said candidate agent is selected from one or more of: small molecule, protein, polypeptide, polysaccharide or polynucleotide, and wherein said candidate agent is identified as an inhibiting modulator of the interaction between STIM and Orai proteins when the candidate agent decreases the energy transfer between CAD or CAD-like proteins and Orai or Orai-derived polypeptides.

24. A composition consisting or consisting essentially of SEQ ID NO:1 or fragments and derivatives thereof.

25. A composition consisting or consisting essentially of SEQ ID NO:2, SEQ ID NO:3, SEQ ID NO:4, or SEQ ID NO:5 or fragments and derivatives thereof.
